(19) United States
(12) Patent Application Publication
(10) Pub. No.: US 2024/0366417 A1
(43) Pub. Date: Nov. 7, 2024

(54) PATIENT INTERFACE WITH MOUTH CLOSURE STRUCTURE

(71) Applicant: ResMed Pty Ltd, Bella Vista (AU)

(72) Inventors: Lachlan Richard GOLDSPINK, Bella Vista (AU); Michiel KOOIJ, Bella Vista (AU); Stewart Joseph WAGNER, Bella Vista (AU)

(21) Appl. No.: 18/653,942

(22) Filed: May 2, 2024

(30) Foreign Application Priority Data
May 4, 2023 (AU) 2023901327

(51) Int. Cl. A61F 5/56; A61M 16/06; A61M 16/16; A61M 16/20

(52) U.S. Cl. CPC A61F 5/56; A61M 16/0616; A61M 16/0683; A61M 16/16; A61M 16/209

(57) ABSTRACT

Forms of the present technology relate to a patient interface for delivering breathable gas to a patient. The patient interface may comprise a pressurisable plenum chamber and a seal-forming structure configured to form a seal with a region of the patient's face surrounding an entrance to the patient's nares but not around the patient's mouth. The patient interface may further comprise a vent structure to allow a continuous flow of gases exhaled by the patient from an interior of the plenum chamber to ambient. The patient interface may further comprise a mouth closure structure configured to promote closure of the patient's mouth. In some forms, the seal-forming structure may comprise at least one adhesive surface configured in use to adhere to a region of the patient's face to form the seal. In some forms, the mouth closure structure may be integrally connected to the seal-forming structure and/or the plenum chamber.

### 1 CROSS-REFERENCE TO RELATED APPLICATIONS

This application claims priority to Australian Provisional Patent Application No. 2023901327 filed May 4, 2023, the entire contents of each of which are hereby incorporated by reference.

### 2 BACKGROUND OF THE TECHNOLOGY

### 2.1 Field of the Technology

The present technology relates to one or more of the screening, diagnosis, monitoring, treatment, prevention and amelioration of respiratory-related disorders. The present technology also relates to medical devices or apparatus, and their use. The present technology relates to seal-forming structures for patient interfaces which form a seal with a patient's airways through adhesive surfaces. The present technology also relates to patient interfaces with mechanisms for promoting closure of the mouth during use.

### 2.2 Description of the Related Art

### 2.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD) and Chest wall disorders.

A range of therapies have been used to treat or ameliorate such conditions. Furthermore, otherwise healthy individuals may take advantage of such therapies to prevent respiratory disorders from arising. However, these have a number of shortcomings.

One of the major issues in respiratory therapy is adherence, which is also referred to as compliance. Usually, a patient may be required to don a patient interface for prolonged periods as part of the respiratory therapy. Bulky and/or obtrusive patient interfaces often lead to patients discontinuing the respiratory therapy due to discomfort, inconvenience or interference with sleep. In particular, it is difficult to ensure that infants and children do not remove patient interface during respiratory therapy.

### 2.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 2.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube or endotracheal tube. In some forms, the comfort and effectiveness of these therapies may be improved.

### 2.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and/or data management.

### 2.2.3.1 Patient Interface

A patient interface may be used to interface respiratory equipment to its wearer, for example by providing a flow of air to an entrance to the airways. The flow of air may be provided to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure.

Conventionally, mask systems are used as patient interfaces to convey the flow of air. These mask systems typically include a plenum chamber which is secured against the patient's face through headgear. The plenum chamber, with the patient's face, encloses a volume of space, which may accommodate the facial features of the patient such as their nose and/or mouth. Often, the plenum chamber may be made of a rigid material. These aspects of the design of some conventional patient interfaces can make sleeping while wearing the patient interface on inconvenient, uncomfortable and potentially claustrophobic for the patient.

Mask systems other than those typically used for respiratory therapy may be functionally unsuitable for the present field. For example, purely ornamental masks may be unable to maintain a suitable pressure. Mask systems used for underwater swimming or diving may be configured to guard against ingress of water from an external higher pressure, but not to maintain air internally at a higher pressure than ambient.

Certain masks may be clinically unfavourable for the present technology e.g. if they block airflow via the nose and only allow it via the mouth.

Certain masks may be impractical for use while sleeping, e.g. for sleeping while lying on one's side in bed with a head on a pillow.

The design of a patient interface presents a number of challenges. The face has a complex three-dimensional shape. The size and shape of noses and heads varies considerably between individuals. Since the head includes bone, cartilage and soft tissue, different regions of the face respond differently to mechanical forces. The jaw or mandible may move relative to other bones of the skull. The whole head may move during the course of a period of respiratory therapy.

As a consequence of these challenges, some masks suffer from being one or more of obtrusive, aesthetically undesirable, costly, poorly fitting, difficult to use, and uncomfortable especially when worn for long periods of time or when a patient is unfamiliar with a system. Wrongly sized masks can give rise to reduced compliance, reduced comfort and poorer patient outcomes. Masks designed solely for aviators, masks designed as part of personal protection equipment (e.g. filter masks), SCUBA masks, or for the administration of anaesthetics may be tolerable for their original application, but nevertheless such masks may be undesirably uncomfortable to be worn for extended periods of time, e.g., several hours. As mentioned earlier, this discomfort may lead to a reduction in patient compliance with therapy. This is even more so if the mask is to be worn during sleep.

CPAP therapy is highly effective to treat certain respiratory disorders, provided patients comply with therapy. If a mask is uncomfortable, or difficult to use a patient may not comply with therapy.

It is often recommended that a patient regularly wash their mask, if a mask is required to be cleaned, or if it is difficult to clean (e.g., difficult to assemble or disassemble), patients may not clean their mask and this may impact on patient compliance.

While a mask for other applications (e.g. aviators) may not be suitable for use in treating sleep disordered breathing, a mask designed for use in treating sleep disordered breathing may be suitable for other applications.

For these reasons, patient interfaces for delivery of CPAP during sleep form a distinct field.

### 2.2.3.1.1 Seal-Forming Structure

Patient interfaces may include a seal-forming structure. Since it is in direct contact with the patient's face, the shape and configuration of the seal-forming structure can have a direct impact the effectiveness and comfort of the patient interface.

A patient interface may be partly characterised according to the design intent of where the seal-forming structure is to engage with the face in use. In one form of patient interface, a seal-forming structure may comprise a first sub-portion to form a seal around the left naris and a second sub-portion to form a seal around the right naris. In one form of patient interface, a seal-forming structure may comprise a single element that surrounds both nares in use. Such single element may be designed to for example overlay an upper lip region and/or a nasal bridge region of a face. These different types of patient interfaces may be known by a variety of names by their manufacturer including nasal cushions, nasal pillows, and nasal puffs.

In one form of patient interface a seal-forming structure may comprise an element that surrounds a mouth region in use, e.g. by forming a seal on a lower lip region of a face. In one form of patient interface, a seal-forming structure may comprise a single element that surrounds both nares and a mouth region in use. These patient interfaces may be referred in the art as oral cushions, oro-nasal cushions or full face cushions.

A seal-forming structure that may be effective in one region of a patient's face may be inappropriate in another region, e.g. because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design is able to fit and be comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface, and is intended to seal against the patient's face when force is applied to the patient interface with the seal-forming structure in confronting engagement with the patient's face. The seal-forming structure may include an air or fluid filled cushion, or a molded or formed surface of a resilient seal element made of an elastomer such as a rubber. With this type of seal-forming structure, if the fit is not adequate, there will be gaps between the seal-forming structure and the face, and additional force will be required to force the patient interface against the face in order to achieve a seal.

Another type of seal-forming structure incorporates a flap seal of thin material positioned about the periphery of the mask so as to provide a self-sealing action against the face of the patient when positive pressure is applied within the mask. Like the previous style of seal forming portion, if the match between the face and the mask is not good, additional force may be required to achieve a seal, or the mask may leak. Furthermore, if the shape of the seal-forming structure does not match that of the patient, it may crease or buckle in use, giving rise to leaks.

Another type of seal-forming structure may comprise a friction-fit element, e.g. for insertion into a naris, however some patients find these uncomfortable.

Another form of seal-forming structure may use adhesive to achieve a seal. A seal formed by an adhesive is usually highly effective with little or no leak for typical therapy pressures (e.g. up to 20 cmH₂O).

Regular application and removal of an adhesive-based seal-forming structure may cause skin trauma or irritation. Moreover, conventional adhesive-based seal-forming structures require cleaning of the area of the skin to which the seal-forming structure is to be adhered before affixing the seal-forming structure. Repeated cleaning, which may include alcohol swabbing, can cause damage to the skin.

Further, affixing an adhesive-based seal-forming structure in or around the nasal region may lead to weakening of adhesion due to moisture from the patient's breath. In other regions of the face, the skin may also release moisture which can loosen adhesion, thereby causing leakages which can lead to ineffective respiratory therapy. In addition, when a patient is subjected to oxygen therapy, leakages can lead to unnecessary loss of the oxygen gas. This leakage of oxygen may be particularly disadvantageous in developed countries where medical oxygen is a scarce and expensive resource.

Adhesive-based seal-forming structures may also leave a residue, odour or colour on the patient's skin, sometimes even after the seal-forming structure is removed.

A range of patient interface seal-forming structure technologies are disclosed in the following patent applications, assigned to ResMed Pty Ltd: WO 1998/004,310; WO 2006/074,513; WO 2010/135,785. Examples of patient interfaces including seal-forming structures which use an adhesive to achieve a seal are disclosed in PCT Publication No. WO 2023/015340, the contents of which are herein incorporated by reference.

One form of nasal pillow is found in the Adam Circuit manufactured by Puritan Bennett. Another nasal pillow, or nasal puff is the subject of U.S. Pat. No. 4,782,832 (Trimble et al.), assigned to Puritan-Bennett Corporation.

ResMed Limited has manufactured the following products that incorporate nasal pillows: SWIFT™ nasal pillows mask, SWIFT™ II nasal pillows mask, SWIFT™ LT nasal pillows mask, SWIFT™ FX nasal pillows mask and MIRAGE LIBERTY™ full-face mask. The following patent applications, assigned to ResMed Limited, describe examples of nasal pillows masks: International Patent Application WO2004/073,778 (describing amongst other things aspects of the ResMed Limited SWIFT™ nasal pillows), US Patent Application 2009/0044808 (describing amongst other things aspects of the ResMed Limited SWIFT™ LT nasal pillows); International Patent Applications WO 2005/063,328 and WO 2006/130,903 (describing amongst other things aspects of the ResMed Limited MIRAGE LIBERTY™ full-face mask); International Patent Application WO 2009/052,560 (describing amongst other things aspects of the ResMed Limited SWIFT™ FX nasal pillows).

### 2.2.3.1.2 Positioning and Stabilising

A seal-forming structure of a patient interface used for positive air pressure therapy is subject to the corresponding force of the air pressure to disrupt a seal. Thus a variety of techniques have been used to position the seal-forming structure, and to maintain it in sealing relation with the appropriate portion of the face.

One technique is the use of adhesives. Examples of patient interfaces which use an adhesive to position and stabilise a seal-forming structure with the face are disclosed in PCT Publication No. WO 2023/015340, the contents of which are herein incorporated by reference. One advantage of the use of adhesives to position and stabilise the seal-forming structure on the patient's face is that it avoids the need for headgear (discussed below), which can be uncomfortable, claustrophobic and adds manufacturing cost and complexity. However, as mentioned before, the use of adhesives, as is known in the art, has some disadvantages.

Another technique is the use of one or more straps and/or stabilising harnesses. Many such harnesses suffer from being one or more of ill-fitting, bulky, uncomfortable and awkward to use. They tend to be less air-tight than adhesive-based seal forming structures. Moreover, straps and/or stabilising harnesses tend to leave markings on the face when used overnight.

### 2.2.3.1.3 Pressurised Air Conduit

In one type of treatment system, a flow of pressurised air is provided to a patient interface through a conduit in an air circuit that fluidly connects to the patient interface so that, when the patient interface is positioned on the patient's face during use, the conduit extends out of the patient interface forwards away from the patient's face. This may sometimes be referred to as a “tube down” configuration.

Conduits connecting to an interface at the front of a patient's face may sometimes be vulnerable to becoming tangled up in bed clothes.

### 2.2.3.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus, RPT devices may also act as flow therapy devices. Examples of RPT devices include a CPAP device and a ventilator.

### 2.2.3.3 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

### 2.2.3.4 Humidifier

Delivery of a flow of air without humidification may cause drying of airways. The use of a humidifier with an RPT device and the patient interface produces humidified gas that minimizes drying of the nasal mucosa and increases patient airway comfort. In addition, in cooler climates, warm air applied generally to the face area in and about the patient interface is more comfortable than cold air.

### 2.2.3.5 Vent Technologies

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

### 2.2.4 Nasal Breathing

It is considered to be advantageous for a patient to keep their mouth closed while asleep. This allows the patient to breathe through their nasal passages. Many patients have a tendency to open their mouths while sleeping, which means that breathing may occur through both the mouth and nasal passages, in some cases reducing the amount of breathing through the nasal passages to relatively low levels. It may be desirable to promote the patient to close their mouth, at least partly, while sleeping.

### 3 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

An aspect of certain forms of the present technology is to provide methods and/or apparatus that improve the compliance of patients with respiratory therapy.

An aspect of one form of the present technology is a patient interface comprising a seal-forming structure which is configured to form a seal with a region of the patient's face surrounding an entrance to the patient's airways.

Another aspect of one form of the present technology is a patient interface comprising a seal-forming structure having an opening such that a flow of breathable gas is delivered to at least an entrance to the patient's nares.

Another aspect of one form of the present technology is a patient interface comprising a seal-forming structure which further comprises at least one adhesive surface configured in use to adhere to the region of the patient's face surrounding the entrance to the patient's airways to form the seal.

An aspect of one form of the present technology is a patient interface comprising a plenum chamber pressurisable to a therapeutic pressure of at least 6 cmH₂O above ambient air pressure, said plenum chamber including a plenum chamber inlet port configured to receive a flow of breathable gas at the therapeutic pressure for breathing by the patient.

In one form of the present technology, the seal-forming structure is configured to maintain said therapeutic pressure in the plenum chamber throughout the patient's respiratory cycle in use.

Another aspect of one form of the present technology is a patient interface that has a perimeter shape which is complementary to that of an intended wearer. In one form, the seal-forming structure is configured to have a perimeter shape which is complementary to the region of the patient's face surrounding the entrance to the patient's airways to form the seal. The regions to which the seal-forming structure is to be adhered to may be referred to as the target sealing regions. In one form, the seal-forming structure is configured such that the region of the patient's face comprises regions of the patient's face adjacent to, or surrounding, the nares.

Another aspect of the present technology is a patient interface comprising a mouth closure structure configured to promote closure of the patient's mouth.

According to one aspect of the technology there is provided a patient interface for use in delivering breathable gas to a patient. The patient interface may comprise a plenum chamber pressurisable to a therapeutic pressure of at least 6 cmH₂O above ambient air pressure. The plenum chamber may include a plenum chamber inlet port configured to receive a flow of breathable gas at the therapeutic pressure for breathing by the patient. The patient interface may further comprise a seal-forming structure provided to the plenum chamber. The seal-forming structure may be configured to form a seal with a region of the patient's face surrounding an entrance to the patient's nares but not around the patient's mouth. The seal-forming structure may have an opening therein such that the flow of breathable gas is delivered to the entrance to the patient's nares. The seal-forming structure may be configured to maintain said therapeutic pressure in the plenum chamber throughout the patient's respiratory cycle in use. The seal-forming structure may comprise at least one adhesive surface configured in use to adhere to a region of the patient's face to form the seal. The patient interface may further comprise a vent structure to allow a continuous flow of gases exhaled by the patient from an interior of the plenum chamber to ambient. The vent structure may be configured to maintain the therapeutic pressure in the plenum chamber in use. The patient interface may further comprise a mouth closure structure configured to promote closure of the patient's mouth.

In certain forms, the mouth closure structure may comprise a mouth closure member comprising an adhesive surface configured in use to adhere to the patient's lips and/or a region of the patient's face proximate the patient's lips.

In certain forms, the mouth closure member may be configured to adhere to a region of the patient's face inferior to the mouth and to a region of the patient's face superior to the mouth.

In certain forms, when the mouth closure member is adhered to the patient's face, the mouth closure member may leave at least a portion of the patient's mouth uncovered. For example, when the mouth closure member is adhered to the patient's face, the mouth closure member may leave the patient's mouth fully uncovered.

In certain forms, the mouth closure member may be configured to adhere to one or more side-of-mouth regions on one or both lateral sides of the patient's mouth and to a lower lip/chin region between the patient's mouth and chin.

In certain forms, the mouth closure member may be configured to span across the patient's mouth.

In certain forms, the mouth closure structure may be integrally connected to the seal-forming structure and/or the plenum chamber.

In certain forms, the mouth closure structure may be configured to be disconnectable from the seal-forming structure and/or the plenum chamber.

According to one aspect of the technology there is provided a patient interface for use in delivering breathable gas to a patient. The patient interface may comprise a plenum chamber pressurisable to a therapeutic pressure of at least 6 cmH₂O above ambient air pressure. The plenum chamber may include a plenum chamber inlet port configured to receive a flow of breathable gas at the therapeutic pressure for breathing by the patient. The patient interface may further comprise a seal-forming structure provided to the plenum chamber. The seal-forming structure may be configured to form a seal with a region of the patient's face surrounding an entrance to the patient's nares but not around the patient's mouth. The seal-forming structure may have an opening therein such that the flow of breathable gas is delivered to the entrance to the patient's nares. The seal-forming structure may be configured to maintain said therapeutic pressure in the plenum chamber throughout the patient's respiratory cycle in use. The patient interface may further comprise a vent structure to allow a continuous flow of gases exhaled by the patient from an interior of the plenum chamber to ambient. The vent structure may be configured to maintain the therapeutic pressure in the plenum chamber in use. The patient interface may further comprise a mouth closure structure configured to promote closure of the patient's mouth. The mouth closure structure may be connected to the seal-forming structure and/or the plenum chamber.

In certain forms, the seal-forming structure may comprise at least one adhesive surface configured in use to adhere to a region of the patient's face to form the seal.

In certain forms, the patient interface may further comprise headgear configured to hold the seal-forming structure in sealing position on the patient's face in use.

In certain forms, the headgear may comprise one or more straps configured to hold the seal-forming structure in sealing position on the patient's face in use.

In certain forms, the mouth closure structure may comprise a mouth closure member comprising an adhesive surface configured in use to adhere to the patient's lips and/or a region of the patient's face proximate the patient's lips.

In certain forms, the mouth closure member may be configured to adhere to a region of the patient's face inferior to the mouth and to a region of the patient's face superior to the mouth.

In certain forms, when the mouth closure member is adhered to the patient's face, the mouth closure member may leave at least a portion of the patient's mouth uncovered. For example, when the mouth closure member is adhered to the patient's face, the mouth closure member may leave the patient's mouth fully uncovered.

In certain forms, the mouth closure member may be configured to adhere to one or more side-of-mouth regions on one or both lateral sides of the patient's mouth and to a lower lip/chin region between the patient's mouth and chin.

In certain forms, the mouth closure member may be configured to span across the patient's mouth.

In certain forms, the mouth closure structure may be directly connected to the seal-forming structure and/or the plenum chamber, for example the mouth closure structure may be integrally connected to the seal-forming structure and/or the plenum chamber.

In certain forms, the mouth closure structure may be configured to be disconnectable from the seal-forming structure and/or the plenum chamber.

An aspect of certain forms of the present technology is a medical device that is easy to use, e.g. by a person who does not have medical training, by a person who has limited dexterity, vision or by a person with limited experience in using this type of medical device.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 4 BRIEF DESCRIPTION OF THE DRAWINGS

The present technology is illustrated by way of example, and not by way of limitation, in the figures of the accompanying drawings, in which like reference numerals refer to similar elements including:

FIG. 1 shows a system including a patient 1000 wearing a patient interface 3000, receiving a supply of air at positive pressure from an RPT device 4000. The patient is sleeping lying on their side.

FIG. 2A shows an overview of a human respiratory system including the nasal and oral cavities, the larynx, vocal folds, oesophagus, trachea, bronchus, lung, alveolar sacs, heart and diaphragm.

FIG. 2B shows a view of a human upper airway including the nasal cavity, nasal bone, lateral nasal cartilage, greater alar cartilage, nostril, lip superior, lip inferior, larynx, hard palate, soft palate, oropharynx, tongue, epiglottis, vocal folds, oesophagus and trachea.

FIG. 2C is a front view of a face with several features of surface anatomy identified including the lip superior, upper vermilion, lower vermilion, lip inferior, mouth width, endocanthion, a nasal ala, nasolabial sulcus and cheilion. Also indicated are the directions superior, inferior, radially inward and radially outward.

FIG. 2D is a side view of a head with several features of surface anatomy identified including glabella, sellion, pronasale, subnasale, lip superior, lip inferior, supramenton, nasal ridge, alar crest point, otobasion superior and otobasion inferior. Also indicated are the directions superior & inferior, and anterior & posterior.

FIG. 2E is a further side view of a head. The approximate locations of the Frankfort horizontal and nasolabial angle are indicated. The coronal plane is also indicated.

FIG. 2F shows a base view of a nose with several features identified including naso-labial sulcus, lip inferior, upper Vermilion, naris, subnasale, columella, pronasale, the major axis of a naris and the midsagittal plane.

FIG. 2G shows a side view of the superficial features of a nose.

FIG. 2H shows subcutaneal structures of the nose, including lateral cartilage, septum cartilage, greater alar cartilage, lesser alar cartilage, sesamoid cartilage, nasal bone, epidermis, adipose tissue, frontal process of the maxilla and fibrofatty tissue.

FIG. 2I shows a medial dissection of a nose, approximately several millimeters from the midsagittal plane, amongst other things showing the septum cartilage and medial crus of greater alar cartilage.

FIG. 2J shows a front view of the bones of a skull including the frontal, nasal and zygomatic bones. Nasal concha are indicated, as are the maxilla, and mandible.

FIG. 2K shows a lateral view of a skull with the outline of the surface of a head, as well as several muscles. The following bones are shown: frontal, sphenoid, nasal, zygomatic, maxilla, mandible, parietal, temporal and occipital. The mental protuberance is indicated. The following muscles are shown: digastricus, masseter, sternocleidomastoid and trapezius.

FIG. 2L shows an anterolateral view of a nose.

FIG. 3 shows a patient interface which is configured to adhere to an alar rim region of a patient's face in accordance with one form of the present technology.

FIG. 4A shows a perspective view of a patient interface which is adhered to a patient's face in accordance with one form of the present technology.

FIG. 4B shows a side view of the patient interface of FIG. 4A.

FIG. 5 a perspective view of a patient interface which is adhered to an alar crease region of a patient's face in accordance with one form of the present technology.

FIG. 6 shows a seal-forming structure in accordance with one form of the present technology.

FIG. 7 is a perspective view illustration of a patient interface in accordance with another form of the present technology.

FIG. 8 illustrates a perspective view of a patient interface according to one form of the technology.

FIG. 9 is a perspective view of a patient interface according to another form of the technology.

FIG. 10 is a perspective view of a patient interface according to another form of the technology.

FIG. 11 is an illustration of a patient interface comprising a mouth closure structure according to one form of the technology.

FIG. 12 is an illustration of a patient interface comprising a mouth closure structure according to another form of the technology.

FIG. 13 is an illustration of a patient interface comprising a mouth closure structure according to another form of the technology.

FIG. 14 is an illustration of a patient interface comprising a mouth closure structure according to another form of the technology.

FIG. 15 is an illustration of part of the patient interface shown in FIG. 14.

FIG. 16 is an illustration of part of the patient interface shown in FIG. 14.

### 5 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 5.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 5.2 Respiratory Therapy Systems

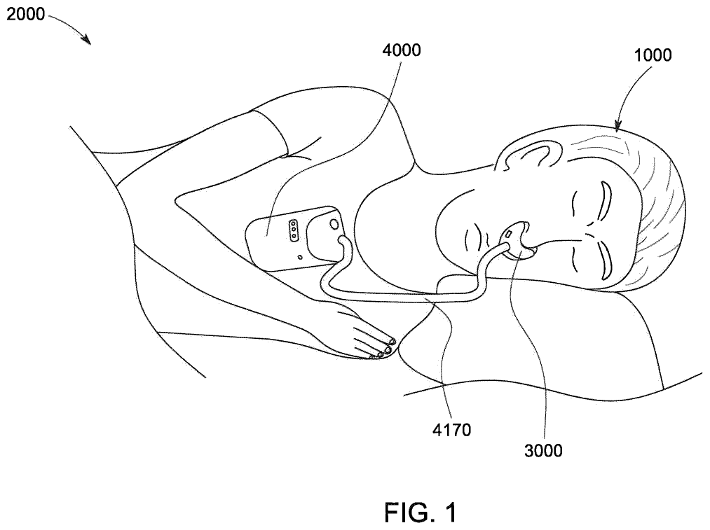

In certain forms, as shown in FIG. 1, the present technology comprises a respiratory therapy system 2000 for treating a respiratory disorder. The respiratory therapy system 2000 may comprise an RPT device 4000 for supplying a flow of air to the patient 1000 via an air circuit 4170 and a patient interface 3000.

In the form of the technology shown in FIG. 1, the RPT device 4000 is portable and can be carried by the patient 1000, for example attached to the patient's clothing. In alternative forms of the present technology (not shown in the FIGS.), the RPT device is configured to rest on a nearby surface during use, for example a bedside table.

Further, the respiratory therapy system 2000 may include a humidifier to change the absolute humidity of air or gas for delivery to a patient relative to ambient air. Typically, the humidifier is used to increase the absolute humidity and increase the temperature of the flow of air (relative to ambient air) before delivery to the patient's airways.

### 5.3 Patient Interface

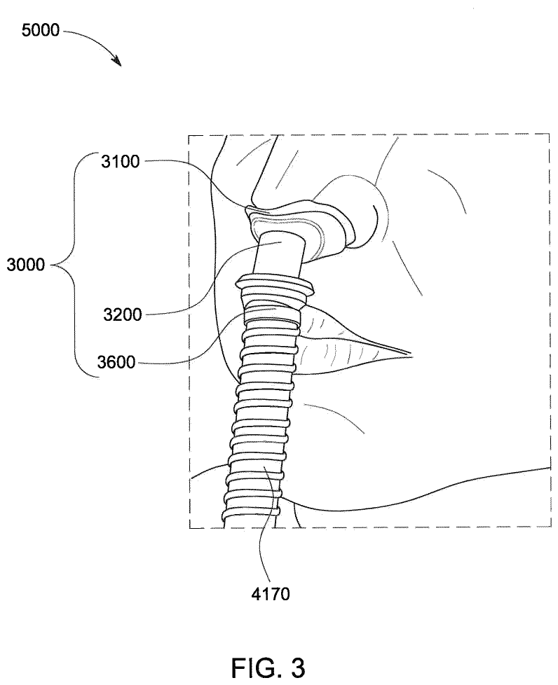

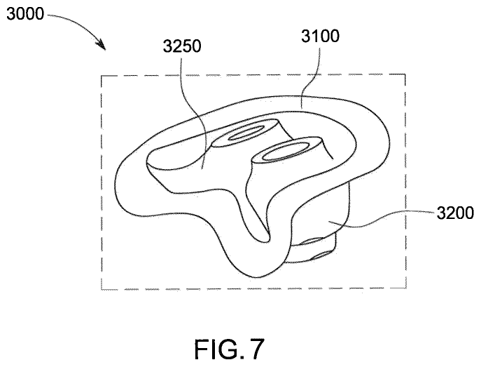

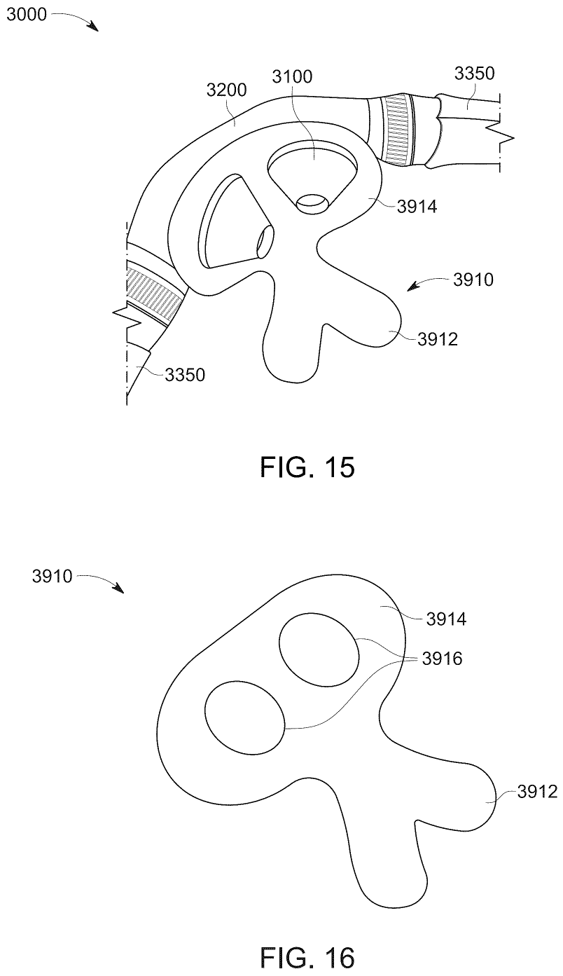

FIGS. 3-6 and 8-13 show forms of the technology providing a patient interface 3000 (or parts thereof) in which the patient interface 3000 does not extend into the nares of the patient 1000. In the form of technology shown in FIGS. 7 and 14-16, patient interface 3000 comprises nasal prongs 3250 that engage with and/or extend into the nares. These types of patient interfaces will be described in more detail in the following paragraphs.

A patient interface 3000, such as shown in FIGS. 3 to 16, in accordance with certain aspects of the present technology comprises at least some of the following functional aspects: a seal-forming structure 3100, a plenum chamber 3200, a positioning and stabilising structure 3300, a vent 3400 and a mouth closure structure 3910.

In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

The plenum chamber 3200 may be formed of one or more modular components (e.g., a cushion module 3150 together with the seal-forming structure 3100) in the sense that it or they can be replaced with different components, for example components of a different size and/or shape.

If a patient interface is unable to comfortably deliver a minimum level of positive pressure to the airways, the patient interface may be unsuitable for respiratory pressure therapy.

The patient interface 3000 in accordance with one form of the present technology is constructed and arranged to be able to provide a supply of air at a positive pressure above the ambient, for example at least 2, 4, 6, 10, or 20 cmH₂O with respect to ambient.

### 5.3.1 Seal-Forming Structure

In one form of the present technology, the patient interface 3000 includes a seal-forming structure 3100 which is configured to form a seal with a region of the patient's face. The seal-forming structure 3100 is thereby configured to secure the plenum chamber 3200 in a sealing engagement with respect to the patient's face. The seal-forming structure 3100 may form an opening to allow a flow of breathable gas to be delivered to at least an entrance to the patient's nares.

In one form of the present technology, the seal-forming structure 3100 provides a target seal-forming region. The target seal-forming region is a region on the seal-forming structure 3100 where sealing may occur. The region where sealing actually occurs—the actual sealing surface—may change within a given treatment session, from day to day, and from patient to patient, depending on a range of factors including for example, where the patient interface was placed on the face, and the shape of a patient's face.

In certain forms of the present technology, the seal-forming structure 3100 is configured so that the shape of the target seal-forming region substantially matches or resembles the shape of the region of the patient's face to which the seal-forming structure 3100 is in use attached, and/or is constructed to be sufficiently flexible that it is able to deform to do so. This may promote a greater degree of sealing against the patient's face and, in the case of a seal-forming structure that adheres to the patient's face, avoids the adhesive surface 3102 pulling on the underlying skin when the patient interface 3000 is in use.

In certain forms of the present technology, the seal-forming structure 3100 is constructed from a biocompatible material, e.g. silicone rubber.

A seal-forming structure 3100 in accordance with the present technology may be constructed from a soft, flexible, resilient material such as silicone or a thermoplastic elastomer (TPE).

In certain forms of the present technology, a system is provided comprising more than one a seal-forming structure 3100, each being configured to correspond to a different size and/or shape range. For example the system may comprise one form of a seal-forming structure 3100 suitable for a large sized head, but not a small sized head and another suitable for a small sized head, but not a large sized head.

More details of seal-forming structure according to certain forms of the technology are described below. Other aspects of seal-forming structures according to forms of the technology are described in more detail in PCT Publication No. WO 2023/015340, the contents of which are herein incorporated by reference.

### 5.3.1.1 Sealing Mechanisms

### 5.3.1.1.1 Adhesive

The seal-forming structure 3100 of certain forms of the technology is configured to adhere, through an adhesive provided on an adhesive surface 3102 of the seal-forming structure 3100, to one or more regions of the patient's face so as to form a seal with a region of the patient's face surrounding an entrance to one or more of the patient's airways. For example, the seal-forming structures 3100 in FIGS. 3 to 8 and 11 to 13 are each configured to seal around the nasal airways of the patient 1000.

The adhesive-based attachment of the seal-forming structure 3100 to the patient's face allows formation of a highly airtight seal. A high quality seal improves the effectiveness of positive pressure respiratory therapy since the desired pressure can be maintained in the patient interface. Furthermore, a high quality seal reduces the overall power required to be expended by an RPT device 4000 to maintain the pressure of the breathable gas in the patient interface 3000. When the seal-forming structure 3100 is adhered to the patient's face, another positioning and stabilising structure, e.g. headgear, may not be provided.

### 5.3.1.1.2 Other Sealing Mechanisms

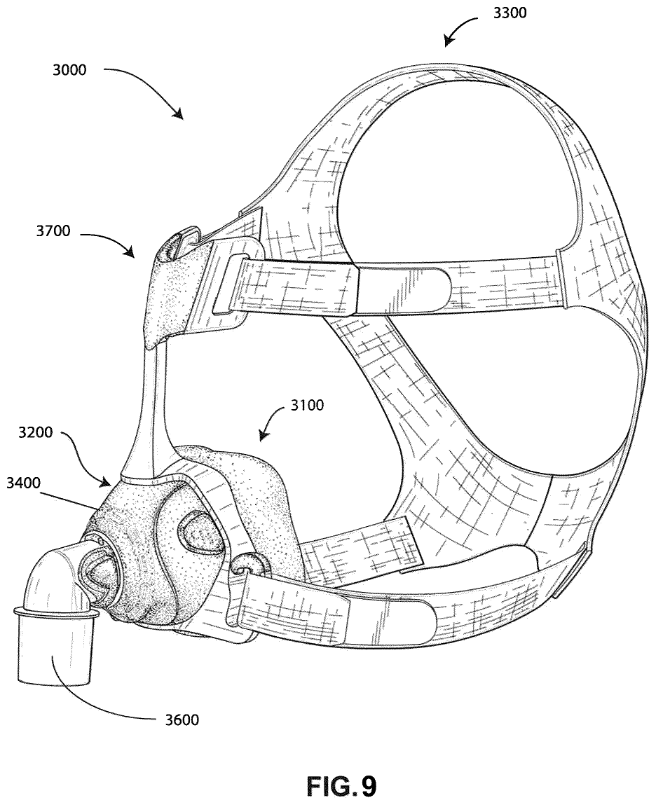

In the forms shown in FIGS. 9, 10 and 14 to 16, the seal-forming structure includes a sealing flange utilizing a pressure assisted sealing mechanism. In use, the sealing flange can readily respond to a system positive pressure in the interior of the plenum chamber 3200 acting on its underside to urge it into tight sealing engagement with the face. The pressure assisted mechanism may act in conjunction with elastic tension in the positioning and stabilising structure.

In one form, the seal-forming structure 3100 comprises a sealing flange and a support flange. The sealing flange comprises a relatively thin member with a thickness of less than about 1 mm, for example about 0.25 mm to about 0.45 mm, which extends around the perimeter of the plenum chamber 3200. Support flange may be relatively thicker than the sealing flange. The support flange is disposed between the sealing flange and the marginal edge of the plenum chamber 3200, and extends at least part of the way around the perimeter. The support flange is or includes a spring-like element and functions to support the sealing flange from buckling in use.

In one form, the seal-forming structure may comprise a compression sealing portion or a gasket sealing portion. In use the compression sealing portion, or the gasket sealing portion is constructed and arranged to be in compression, e.g. as a result of elastic tension in the positioning and stabilising structure 3300.

In one form, the seal-forming structure comprises a tension portion. In use, the tension portion is held in tension, e.g. by adjacent regions of the sealing flange.

In certain forms of the present technology, a seal-forming structure may comprise one or more of a pressure-assisted sealing flange, a compression sealing portion, a gasket sealing portion, a tension portion, and a portion having a tacky or adhesive surface.

### 5.3.1.2 Sealing Region of Patient's Face

In certain forms of the technology, the seal-forming structure 3100 forms a seal in use with a region of the patient's face surrounding an entrance of the patient's nares. In certain forms, the seal-forming structure 3100 forms a seal around an entrance to the patient's nasal airways (i.e. one or both nares) but not around the patient's mouth.

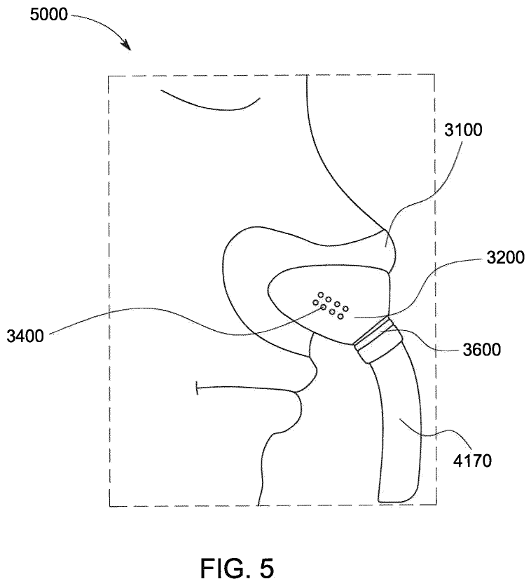

In the exemplary forms of technology shown in FIGS. 5, 9 and 10, the seal-forming structure 3100 may be configured to seal to the patient's lip superior. The patient interface 3000 may leave the patient's mouth uncovered. This patient interface 3000 may deliver a supply of air or breathable gas to both nares of patient 1000 and not to the mouth. This type of patient interface may be identified as a nose-only mask.

One form of nose-only mask according to the present technology is what has traditionally been identified as a “nasal mask”, having a seal-forming structure 3100 configured to seal on the patient's face around the nose and over the bridge of the nose. A nasal mask may be generally triangular in shape. In one form, the non-invasive patient interface 3000 comprises a seal-forming structure 3100 that forms a seal in use to an upper lip region (e.g. the lip superior), to the patient's nose bridge or at least a portion of the nose ridge above the pronasale, and to the patient's face on each lateral side of the patient's nose, for example proximate the patient's nasolabial sulci. The patient interface 3000 shown in FIG. 9 has this type of seal-forming structure 3100. This patient interface 3000 may deliver a supply of air or breathable gas to both nares of patient 1000 through a single orifice.

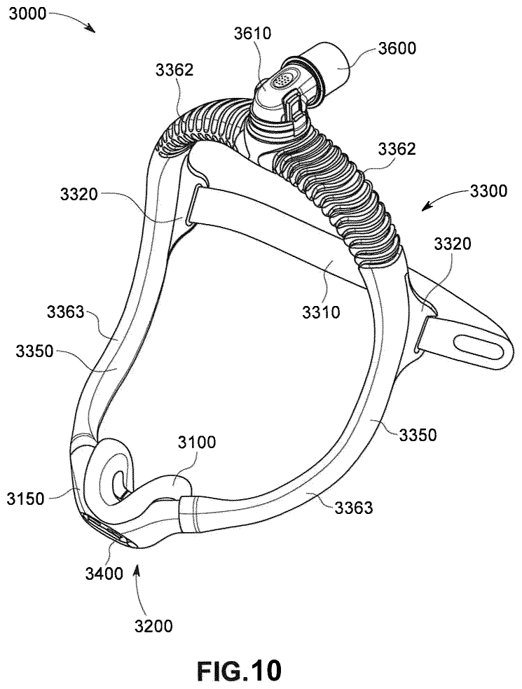

Another form of nose-only mask may seal around an inferior periphery of the patient's nose without engaging the user's nasal ridge. This type of patient interface 3000 may be identified as a “nasal cradle” mask and the seal-forming structure 3100 may be identified as a “nasal cradle cushion”, for example. In one form, for example as shown in FIG. 10, the seal-forming structure 3100 is configured to form a seal in use with inferior surfaces of the nose around the nares. The seal-forming structure 3100 may be configured to seal around the patient's nares at an inferior periphery of the patient's nose including to an inferior and/or anterior surface of a pronasale region of the patient's nose and to the patient's nasal alae. The seal-forming structure 3100 may seal to the patient's lip superior. The shape of the seal-forming structure 3100 may be configured to match or closely follow the underside of the patient's nose and may not contact a nasal bridge region of the patient's nose or any portion of the patient's nose superior to the pronasale. In one form of nasal cradle cushion, the seal-forming structure 3100 comprises a bridge portion dividing the opening into two orifices, each of which, in use, supplies air or breathable gas to a respective one of the patient's nares. The bridge portion may be configured to contact or seal against the patient's columella in use. Alternatively, the seal-forming structure 3100 may comprise a single opening to provide a flow or air or breathable gas to both of the patient's nares.

In the form shown in FIGS. 7 and 14 to 16, the seal-forming structure of the non-invasive patient interface 3000 comprises a pair of nasal puffs, or nasal pillows, each nasal puff or nasal pillow being constructed and arranged to form a seal with a respective naris of the nose of a patient. Nasal pillows in accordance with an aspect of the present technology include: a frusto-cone, at least a portion of which forms a seal on an underside of the patient's nose, a stalk, a flexible region on the underside of the frusto-cone and connecting the frusto-cone to the stalk. In addition, the structure to which the nasal pillow of the present technology is connected includes a flexible region adjacent the base of the stalk. The flexible regions can act in concert to facilitate a universal joint structure that is accommodating of relative movement both displacement and angular of the frusto-cone and the structure to which the nasal pillow is connected. For example, the frusto-cone may be axially displaced towards the structure to which the stalk is connected.

A more detailed description of regions of the patient's face to which a seal-forming structure 3100 seals during use will now be described in the case of certain forms of the technology in which the seal-forming structure is configured to adhere to the patient's face during use.

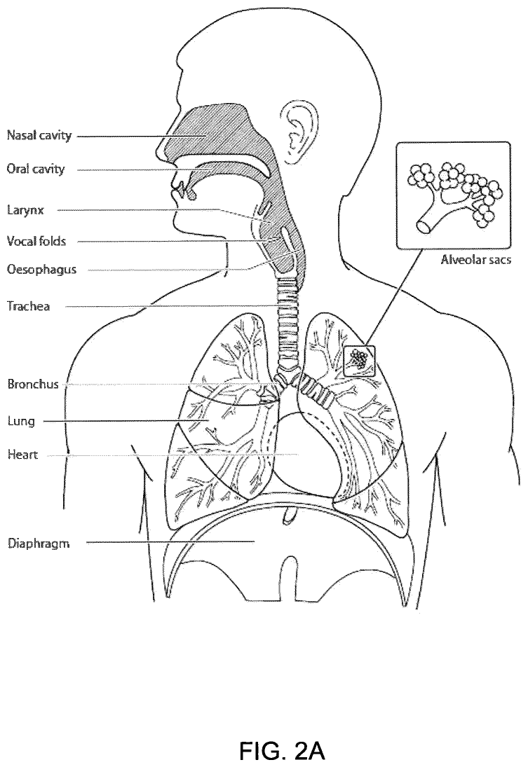

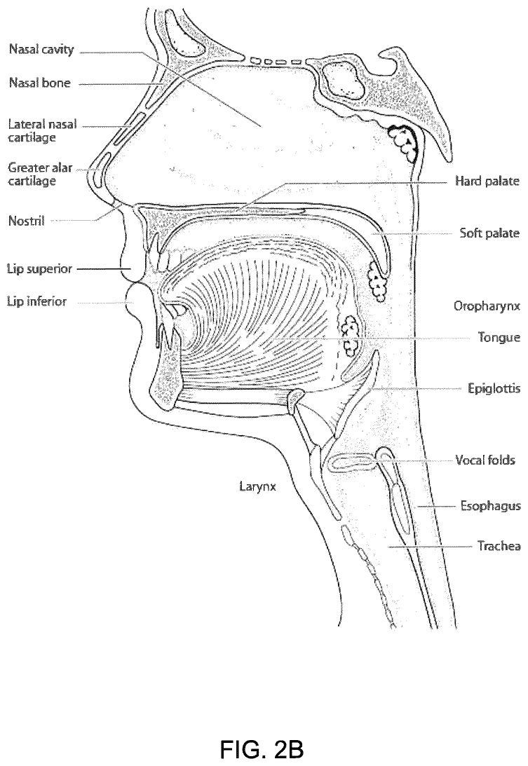

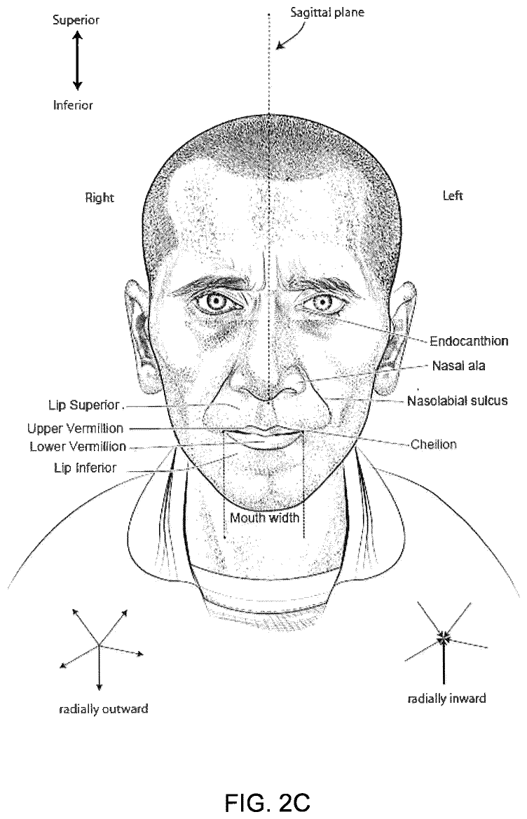

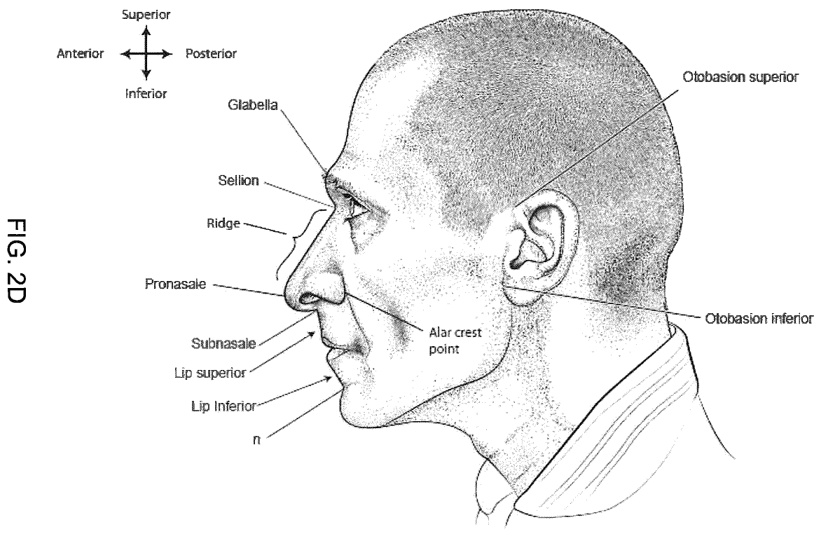

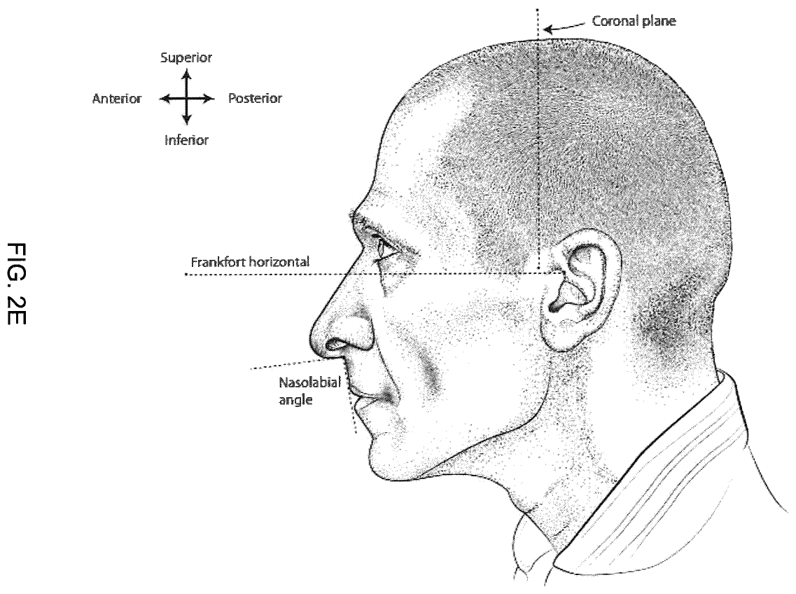

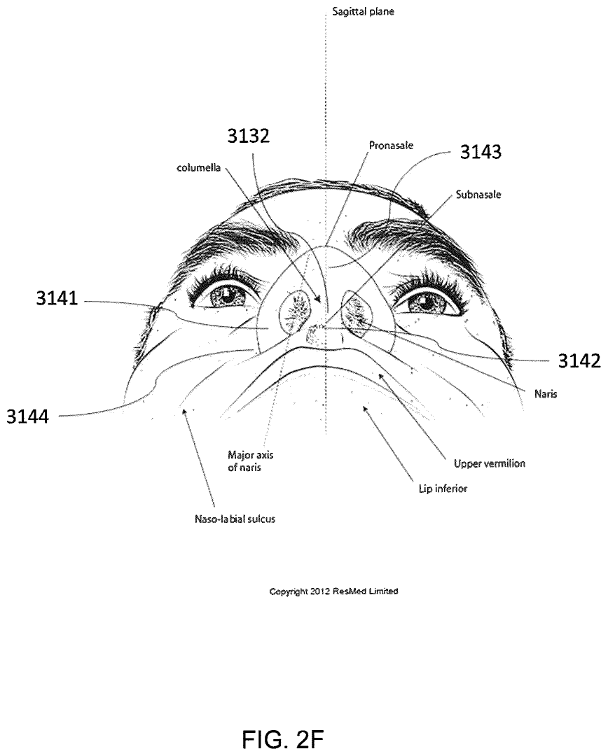

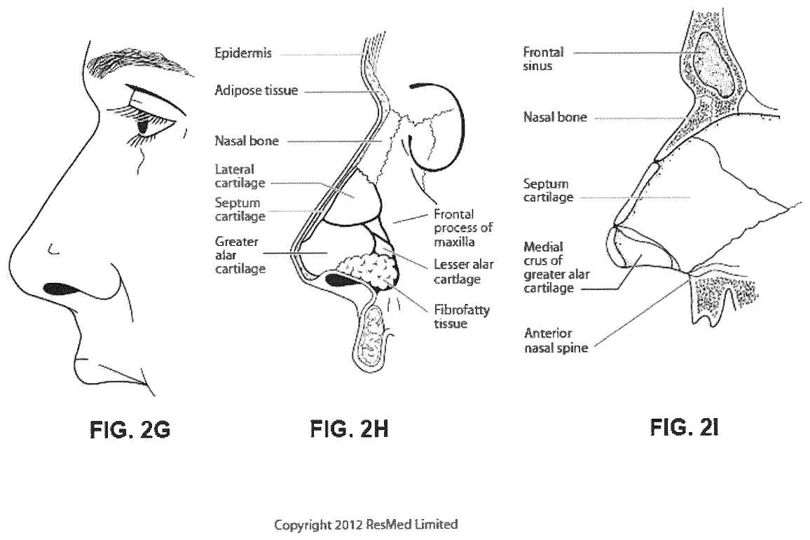

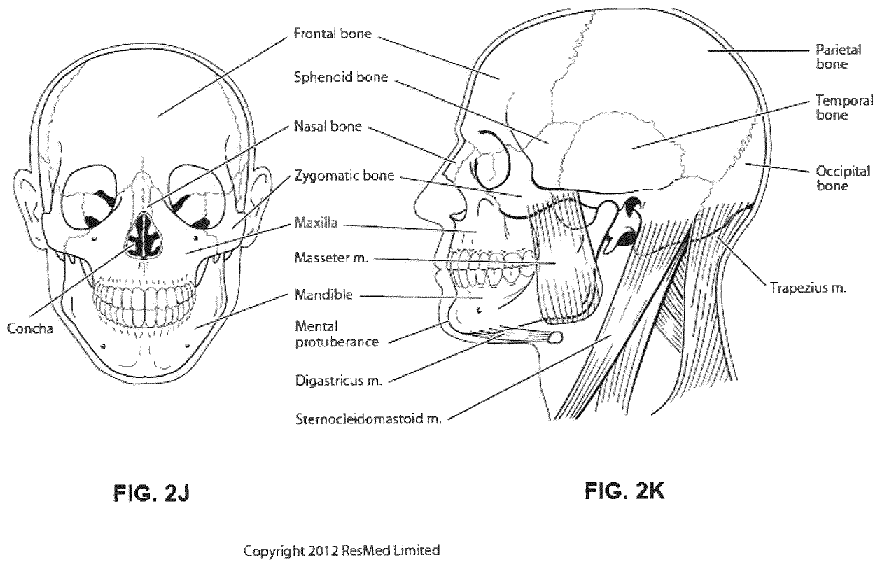

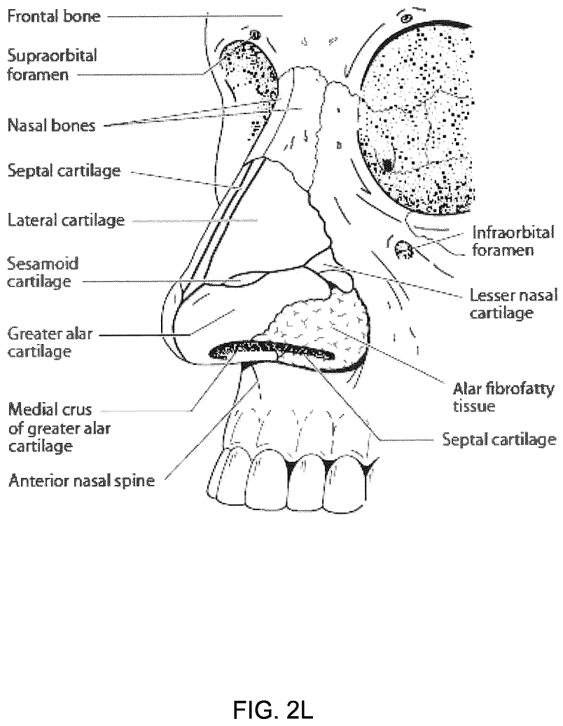

In the form of the technology shown in FIG. 3, the seal-forming structure 3100 is configured to adhere to regions of the patient's face immediately surrounding the nares. These regions may comprise (see FIG. 2F): the alar rim region 3141 (i.e. regions of the ala that are immediately adjacent the nares and may be generally inferiorly facing); the superior-most region of the lip superior 3142, which may comprise the subnasale and/or the region immediately inferior of the subnasale; and an anterior region of the nose that is inferior, e.g. immediately inferior, to the pronasale 3143. In the lateral direction, the seal-forming structure 3100 extends to a region 3144 slightly inferior to the alar crest point, for example a region immediately medial to the junction between the alar crest point and the nasolabial sulcus. In the form of the technology shown, the seal-forming structure 3100 does not adhere to a significant part of the side regions of the nasal alar, although in some forms, or for some faces, it may adhere to the inferior regions of the side regions of the nasal alar. Furthermore, the seal-forming structure 3100 of FIG. 3 does not adhere to the pronasale.

In the form of the technology shown in FIG. 3, the region of the patient's face to which the seal-forming structure 3100 adheres is a band entirely surrounding both the patient's nares. The band may be approximately constant in width around the perimeter of the band.

The region of the face covered by the seal-forming structure 3100 in the form of the technology shown in FIG. 3 has been found not to change shape substantially when a patient 1000 changes their position because this facial region predominantly comprises of cartilage and bone and has relatively little adipose tissue, as compared to the check or chin areas. This facial region is also typically free of facial hair or has very little facial hair (for example, some facial hair may be present on the superior-most region of the lip superior 3142). A seal-forming structure 3100 that adheres to this region may be particularly advantageous for a patient 1000 with upper lip hair.

The region of the face covered by the seal-forming structure 3100 in the form of the technology shown in FIG. 3 has also been found to have relatively little variation in shape across patients in representative population samples, including patients of a variety of races.

Further, the size of this region (which may be referred to as the alar rim region) is small in size since it immediately surrounds the nares.

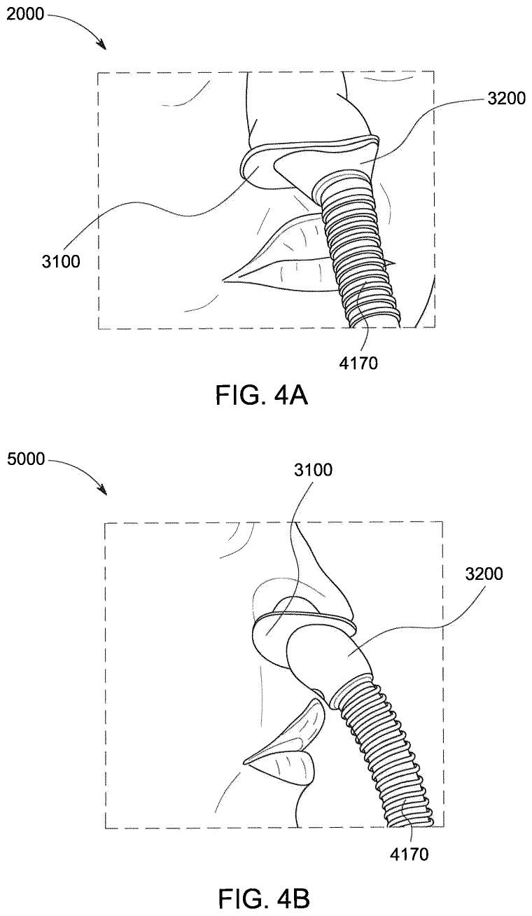

In another exemplary form, as shown in FIGS. 4A and 4B, the seal-forming structure 3100 is configured to adhere to a facial region that extends more superiorly compared to the region shown in FIG. 3 and includes the side regions of the nasal alar. In some forms, the seal-forming structure 3100 is configured to adhere to a side region of the nasal alar and may also extend radially outwardly sufficiently far to adhere in use to regions of the cheeks adjacent the nasal alar crest point, e.g. the regions between the nasal alar and the nasolabial sulcus. This seal-forming structure 3100 may also adhere to the regions of the face to which the seal-forming structure of FIG. 3 adheres. The larger area of adhesion of the seal-forming structure in FIGS. 4A and 4B may improve the amount of adhesion and result in fewer leaks compared to the seal-forming structure of FIG. 3, but may result in more discomfort for the patient.

In another exemplary form, as shown in FIG. 5, the seal-forming structure 3100 is configured to adhere to a larger region of the patient's face than the region to which the seal-forming structures of FIGS. 3, 4A and 4B adhere. In this form the seal-forming structure adheres to a region that extends in a superior direction up to the alar crease region, i.e. when the seal-forming structure is adhered to the patient's face, a superior-most portion of the seal-forming structure adheres to the patient's alar crease. The seal-forming structure 3100 in this form may additionally, or alternatively, adhere to a substantial portion of the lip superior. Furthermore, the seal-forming structure 3100 in this form may additionally, or alternatively, adhere to the pronasale region, which may include a point which is immediately superior to the pronasale. In the lateral direction, the seal-forming structure 3100 in this form adheres to a region of the patient's cheeks adjacent to the nasal alar, including the regions between the nasal alar and the nasolabial sulcus, and the seal-forming structure 3100 may cover the nasal alar completely in use. This seal-forming structure 3100 may also adhere to the regions of the face to which the seal-forming structures of FIGS. 3, 4A and/or 4B adhere. Alternatively, the seal-forming structure of FIG. 5 may be configured to adhere to some regions positioned radially outwardly from the nares compared to some regions to which the seal-forming structures of FIGS. 3, 4A and/or 4B adhere.

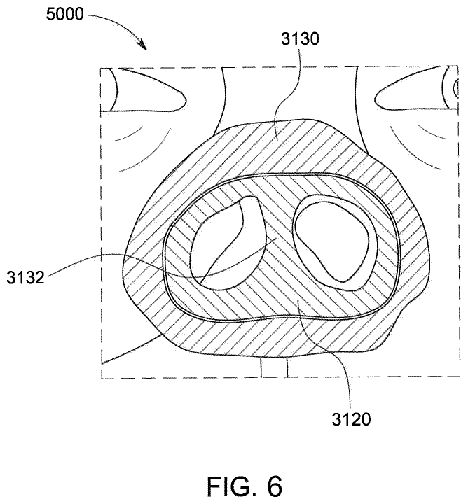

In certain forms of the technology, for example the form of the technology shown in FIG. 6, the seal-forming structure 3100 may be configured to adhere to the columella. For example, the seal-forming structure 3100 may comprise a septum region 3132 extending between two diametrically opposing regions of the seal-forming structure 3100, the septum region 3132 being configured to adhere in use to the columella.

### 5.3.1.3 Composition of the Seal-Forming Structure

In certain forms of the present technology, the seal-forming structure 3100 is constructed from a material having one or more of the following properties: biocompatibility; soft; flexible; stretchable and optionally resilient. In exemplary forms of the technology, the seal-forming structure 3100 is formed from silicone or a thermoplastic elastomer (TPE). In other forms, the seal-forming structure 3100 is formed from a textile, fabric and/or foam material.

In the case of forms of the technology in which an adhesive is used to adhere the seal-forming structure 3100 to the patient's face, any form of adhesive may be used, and adhesives may be applied to any suitable substrate, which may include materials such as mentioned above, for example silicone or TPE. For example, a rubber zinc oxide adhesive may be used. In other forms, other adhesives may be used, for example acrylic or acrylate adhesives, or silicone adhesives.

The adhesives may be provided in the form of adhesive tapes, in which the adhesive is already provided on a substrate (i.e. the tape), which may be advantageously used as the seal-forming structure 3100, or part thereof, or may be readily attached to the seal-forming structure 3100. Examples of suitable tapes are the 3M™ Nexcare™ tape and Leukoplast tape. In some forms, the material may comprise a rayon substrate to which an adhesive is applied. In one form, the seal-forming structure may be formed from, or may comprise, 3M™ Product No. 2484 (which uses a silicone adhesive, “Hi-Tack 3M medical silicone adhesive”), 3M™ Medical tape 9833 (which uses an acrylic/acrylate adhesive), or a similar type of product or a product having a similar structure. Multiple layers of a tape or product may be used to form the seal-forming structure 3100.

In some forms, a fluid adhesive may be applied to a surface of the seal-forming structure 3100, for example in the form of a spray.

In some forms, the seal-forming structure 3100 may be attached to a patient's face using one or more action-release adhesives. The action-release adhesive(s) may be configured so that its adhesive strength is reduced when an “action” is effected. The reduction in the adhesion strength may be sufficient to allow the seal-forming structure 3100 to be easily removed from the patient's face while causing an acceptable level of discomfort. In some forms, the action may be some change or effect that is applied to the adhesive, or to a component (such as the seal-forming structure 3100) to which the adhesive is applied. For example, the patient interface 3000 may comprise a seal-forming structure 3100 having an adhesive surface 3102 where the adhesive strength of the adhesive surface 3102 can be reduced by the deformation of the adhesive surface 3102. The deformation may be a stretch action, i.e. the adhesive surface 3102 may be provided with, or formed from, a stretch-release adhesive. A stretch-release adhesive may have certain adhesive properties only when the adhesive surface 3102 is substantially unstretched. The adhesive surface 3102 may be configured to have reduced and/or no adhesive properties when the adhesive surface 3102 is stretched. In certain forms, acrylate-based adhesives may be used as a stretch-release adhesive for adhesive surface 3102, for example Fixomull™ or 3M™ Stretch Release Tape.

### 5.3.2 Plenum Chamber

The plenum chamber 3200 of certain forms of the technology is configured to receive the flow of breathable gas at the therapeutic pressure for breathing by the patient from the air circuit 4170. The plenum chamber may be formed to be pressurisable to a therapeutic pressure of at least 6 cmH₂O above ambient air pressure, and up to pressures of around 20 cmH₂O or 30 cmH₂O in certain forms.

In one form, the plenum chamber 3200 has a perimeter that is shaped to be complementary to the surface contour of the face of an average person in the region where a seal will form in use. The complementary shape of the perimeter of the plenum chamber 3200 may be configured to facilitate correct positioning of the patient interface 3000 against the patient's face in use.

Alternatively, in certain forms, the plenum chamber 3200 may be shaped in a customised way to an individual patient. Alternatively, the plenum chamber 3200 of a patient interface 3000 may be selected from one of a plurality of possible forms of plenum chamber 3200, with the appropriate plenum chamber for an individual patient being selected as being most suitable for them.

In use, a marginal edge of the plenum chamber 3200 is positioned in close proximity to an adjacent surface of the face. Actual contact with the face may be provided by the seal-forming structure 3100. The seal-forming structure 3100 may extend in use about the entire perimeter of the plenum chamber 3200.

The plenum chamber 3200 may include at least two openings. One opening, which may be formed in a patient-facing, or posterior, side of the plenum chamber 3200, allows pressurised gas to flow from the internal volume of the plenum chamber 3200 to the patient's airways through the seal-forming structure 3100. This opening may also allow exhaled gas from the patient to flow into the plenum chamber 3200. Another opening, which may be referred to as the plenum chamber inlet port 3202, is configured to allow the flow of breathable gas from the air circuit 4170 into the plenum chamber 3200. In certain forms, the plenum chamber inlet port 3202 may be disposed on a side of the plenum chamber 3200 facing away from the patient in use, i.e. an anterior side of the plenum chamber 3200. In other forms, the patient interface may comprise one or more plenum chamber inlet ports 3202 disposed on lateral sides (e.g. left and right sides) of the plenum chamber 3200.

In certain forms of the present technology, the plenum chamber 3200 is constructed from a translucent material. The use of a translucent material can reduce the obtrusiveness of the patient interface, and help improve compliance with therapy.

In some forms, the plenum chamber 3200 is constructed from a rigid material such as polycarbonate. The rigid material may provide support to the seal-forming structure.

In some forms, the plenum chamber 3200 is constructed from a flexible material (e.g., constructed from a soft, flexible, resilient material like silicone, textile, foam, etc.). For example, in examples then may be formed from a material which has a Young's modulus of 0.4 GPa or lower, for example foam. In some forms of the technology the plenum chamber 3200 may be made from a material having Young's modulus of 0.1 GPa or lower, for example rubber. In other forms of the technology the plenum chamber 3200 may be made from a material having a Young's modulus of 0.7 MPa or less, for example between 0.7 MPa and 0.3 MPa. An example of such a material is silicone.

In some forms, the plenum chamber 3200 and the seal-forming structure 3100 are formed from a single homogeneous piece of material, for example silicone or TPE.

### 5.3.3 Positioning and Stabilising Structure

The seal-forming structure 3100 of the patient interface 3000 of the present technology may be held in sealing position in use by the positioning and stabilising structure 3300. The positioning and stabilising structure 3300 may comprise and function as “headgear” since it engages the patient's head in order to hold the patient interface 3000 in a sealing position. Examples of positioning and stabilising structures are shown in FIGS. 9 and 10.

In one form the positioning and stabilising structure 3300 provides a retention force at least sufficient to overcome the effect of the positive pressure in the plenum chamber 3200 to lift off the face.

In one form the positioning and stabilising structure 3300 provides a retention force to overcome the effect of the gravitational force on the patient interface 3000.

In one form the positioning and stabilising structure 3300 provides a retention force as a safety margin to overcome the potential effect of disrupting forces on the patient interface 3000, such as from tube drag, or accidental interference with the patient interface.

In one form of the present technology, a positioning and stabilising structure 3300 is provided that is configured in a manner consistent with being worn by a patient while sleeping. In one example the positioning and stabilising structure 3300 has a low profile, or cross-sectional thickness, to reduce the perceived or actual bulk of the apparatus. In one form of the present technology, a positioning and stabilising structure 3300 is provided that is configured so as not to be too large and bulky to prevent the patient from lying in a supine sleeping position with a back region of the patient's head on a pillow. In one form of the present technology, a positioning and stabilising structure 3300 is provided that is configured so as not to be too large and bulky to prevent the patient from lying in a side sleeping position with a side region of the patient's head on a pillow.

As has already been explained, in certain forms of the technology, the patient interface 3000 comprises a seal-forming structure 3100 that is configured to adhere to the patient's face so as to form a seal and to maintain the patient interface 3000 in position on the patient's face. In such forms, the seal-forming structure 3100 may be considered to additionally function as the positioning and stabilising structure 3300 through the action of the adhesive.

In other forms, the patient interface 3000 may comprise another form of positioning and stabilising structure 3300. For example, in some forms, the positioning and stabilising structure 3300 may include headgear 3302 with one or more straps which may be worn by the patient in order to assist in properly orienting the seal-forming structure 3100 against the patient's face (e.g., in order to limit or prevent leaks).

Some forms of the headgear 3302 may be constructed from a textile material, which may be comfortable against the patient's skin. The textile may be flexible in order to conform to a variety of facial contours. Although the textile may include rigidisers along a selected length, which may limit bending, flexing, and/or stretching of the headgear 3302.

In certain forms, the headgear 3302 may be at least partially extensible. For example, the headgear 3302 may include elastic, or a similar extensible material. For example, the entire headgear 3302 may be extensible or selected portions may be extensible (or more extensible than surrounding portions). This may allow the headgear 3302 to stretch while under tension, which may assist in providing a sealing force for the seal-forming structure 3100.

In the exemplary form of FIG. 10, the headgear 3302 may be a two-point connection headgear. This means that the headgear 3302 may connect to two separate places.

In certain forms, such as shown in FIG. 9, the headgear 3302 may comprise a strap or straps constructed and arranged so that in use at least a portion of an inferior edge thereof passes superior to an otobasion superior of the patient's head and overlays a portion of a parietal bone without overlaying the occipital bone. In certain forms, the headgear 3302 may additionally or alternatively comprise a strap or straps constructed and arranged so that in use at least a portion of a superior edge thereof passes inferior to an otobasion inferior of the patient's head and overlays or lies inferior to the occipital bone of the patient's head. One or more additional straps may also be provided, for example to interconnect other straps to reduce the tendency for the other straps to move apart during use.

### 5.3.3.1 Conduit Headgear

In some forms of the present technology, the positioning and stabilising structure 3300 comprises one or more headgear tubes 3350 that deliver pressurised air received from a conduit forming part of the air circuit 4170 from the RPT device to the patient's airways, for example through the plenum chamber 3200 and seal-forming structure 3100. In the form of the present technology illustrated in FIG. 10, the positioning and stabilising structure 3300 comprises two tubes 3350 that deliver air to the plenum chamber 3200 from the air circuit 4170. The tubes 3350 are configured to position and stabilise the seal-forming structure 3100 of the patient interface 3000 at the appropriate part of the patient's face (for example, the nose and/or mouth) in use. This allows the conduit of air circuit 4170 providing the flow of pressurised air to connect to a connection port 3600 of the patient interface in a position other than in front of the patient's face, for example on top of the patient's head.

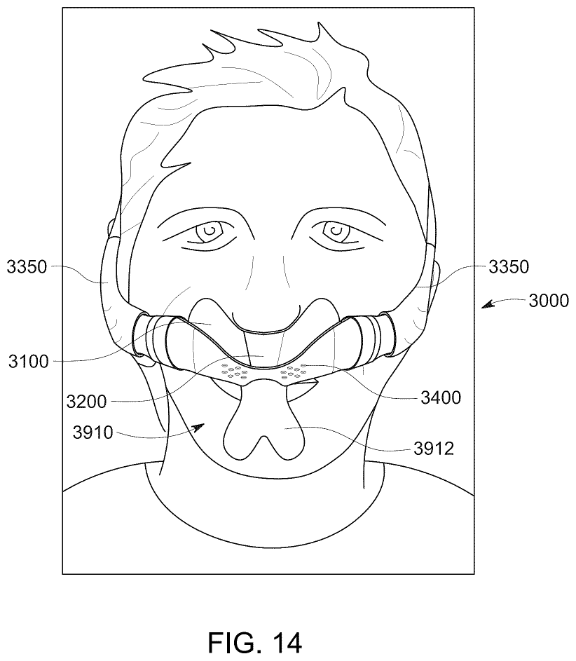

In the form of the present technology illustrated in FIGS. 10 and 14, the positioning and stabilising structure 3300 comprises two tubes 3350, each tube 3350 being positioned in use on a different side of the patient's head and extending across the respective cheek region, above the respective ear (superior to the otobasion superior on the patient's head) to the elbow 3610 on top of the head of the patient 1000. This form of technology may be advantageous because, if a patient sleeps with their head on its side and one of the tubes 3350 is compressed to block or partially block the flow of gas along the tube 3350, the other tube 3350 remains open to supply pressurised gas to the patient. In other examples of the technology, the patient interface 3000 may comprise a different number of tubes, for example one tube, or two or more tubes.

In one form, the tube 3350 may be at least partially extensible so that the tube 3350 and the strap may adjust substantially equal lengths when worn by a patient. This may allow for substantially symmetrical adjustments between the tube 3350 and the strap so that the seal-forming structure remains substantially in the middle. For example, the patient interface 3000 shown in FIG. 10 comprises tubes 3350, the superior portions of which comprise extendable tube sections each in the form of an extendable concertina structure 3362. The inferior portions of tubs 3350 may be non-extendable tube sections 3363.

In the forms of the technology shown in FIGS. 10 and 14, the two tubes 3350 are fluidly connected at superior ends to each other and to the connection port 3600.

The tubes 3350 may be formed from a flexible material, such as an elastomer, e.g. silicone or TPE, and/or from one or more textile and/or foam materials. The tubes 3350 may have a preformed shape and may be able to be bent or moved into another shape upon application of a force but may return to the original preformed shape in the absence of said force. The tubes 3350 may be generally arcuate or curved in a shape approximating the contours of a patient's head between the top of the head and the nasal or oral region.

Each tube 3350 may be configured to receive a flow of air from the connection port 3600 on top of the patient's head and to deliver the flow of air to the seal-forming structure 3100 at the entrance of the patient's airways. In the example shown in FIG. 10, each tube 3350 lies in use on a path extending from the plenum chamber 3200 across the patient's cheek region and superior to the patient's ear to the elbow 3610. For example, a portion of each tube 3350 proximate the plenum chamber 3200 may overlie a maxilla region of the patient's head in use. Another portion of each tube 3350 may overlie a region of the patient's head superior to an otobasion superior of the patient's head. Each of the tubes 3350 may also lie over the patient's sphenoid bone and/or temporal bone and either or both of the patient's frontal bone and parietal bone. The elbow 3610 may be located in use over the patient's parietal bone, over the frontal bone and/or over the junction therebetween (e.g. the coronal suture).

In certain forms of the present technology, the positioning and stabilising structure 3300 comprises at least one headgear strap acting in addition to the tubes 3350 to position and stabilise the seal-forming structure 3100 at the entrance to the patient's airways. In the example shown in FIG. 10, strap 3310 of the positioning and stabilising structure 3300 is connected between the two tubes 3350 positioned on each side of the patient's head and passing around the back of the patient's head, for example overlying or lying inferior to the occipital bone of the patient's head in use. The strap 3310 connects to each tube above the patient's ears. With reference to FIG. 10, the positioning and stabilising structure 3300 comprises a pair of tabs 3320. In use a strap 3310 may be connected between the tabs 3320. The strap 3310 may be sufficiently flexible to pass around the back of the patient's head and lie comfortably against the patient's head, even when under tension in use. In other examples of the present technology, one or more further straps may be provided. For example, patient interfaces 3000 according to examples of the present technology having a nose-and-mouth cushion may have a second, lower, strap configured to lie against the patient's head proximate the patient's neck and/or against posterior surfaces of the patient's neck.

As described above, in some examples of the present technology the patient interface 3000 comprises a seal-forming structure 3100 in the form of a cradle cushion which lies generally under the nose and seals to an inferior periphery of the nose (e.g. an under-the-nose cushion). The positioning and stabilising structure 3300, including the tubes 3350 may be structured and arranged to pull the seal-forming structure 3100 into the patient's face under the nose with a sealing force in a posterior and superior direction (e.g. a posterosuperior direction). A sealing force with a posterosuperior direction may cause the seal-forming structure 3100 to form a good seal to both the inferior periphery of the patient's nose and anterior-facing surfaces of the patient's face, for example on either side of the patient's nose and the patient's lip superior.

### 5.3.4 Forehead Support

In certain forms, for example as shown in FIG. 9, the patient interface 3000 may include a forehead support 3700.

### 5.3.5 Vent

In certain forms of the technology, the patient interface 3000 comprises a vent 3400 constructed and arranged to allow for the washout of exhaled gases, e.g. carbon dioxide. The vent 3400 may be implemented through a vent structure, which may be formed or provided in any one or more components of the patient interface 3000.

In certain forms the vent 3400 is configured to allow a continuous vent flow from an interior of the plenum chamber 3200 to ambient whilst the pressure within the plenum chamber is positive with respect to ambient. The vent 3400 is configured such that the vent flow rate has a magnitude sufficient to reduce rebreathing of exhaled CO₂ by the patient while maintaining the therapeutic pressure in the plenum chamber in use.

One form of vent 3400 in accordance with the present technology comprises a plurality of holes, for example, about 5 to about 80 holes, or about 10 to about 40 holes, or about 20 to about 25 holes.

In certain forms of the technology, for example as shown in FIGS. 5 and 9, the vent 3400 may be located in the plenum chamber 3200.

Alternatively, the vent 3400 may be located in the air circuit 4170 that delivers the flow of breathable gas from the RPT device 4000 to the plenum chamber 3200, for example in a part of the air circuit 4170 located proximate to the plenum chamber 3200.

### 5.3.5.1 Ports

In certain forms of the present technology, a patient interface 3000 includes one or more ports that allow access to the volume within the plenum chamber 3200. In certain forms this allows a clinician to supply supplementary oxygen. In one form, this allows for the direct measurement of a property of gases within the plenum chamber 3200, such as the pressure.

### 5.3.5.2 Breath-to-Atmosphere Vent

In certain forms of the technology, the patient interface 3000 may include a vent 3400 configured to be able to adopt at least two configurations. In one configuration, which may be termed an open configuration, the vent 3400 allows the patient to inhale and exhale through the vent 3400 without significant impedance, or with a level of impedance that is largely unnoticeable by the patient. In another configuration, which may be termed a closed configuration, the vent 3400 is more occluded than in the open configuration. In some forms, in the closed configuration, the vent 3400 allows the washout of exhaled gases from an interior of the plenum chamber 3200 to ambient whilst substantially maintaining the pressure within the plenum chamber as positive with respect to ambient. In other forms, in the closed configuration, the vent may substantially block all washout of gases through the vent, and instead exhaled gases exhaust through a separate vent structure. Such a vent 3400 may be referred to as a “breathe-to-atmosphere” vent (BTA vent).

Whether the BTA vent adopts the open or closed configuration may be based on the pressure of the supply of breathable gas provided from the RPT device 4000 to the patient interface 3000. When no breathable gas is supplied, or when the flow of breathable gas is supplied at a pressure below a certain threshold, for example below a therapeutic pressure level such as 6 cmH₂O, the BTA vent may be configured to adopt the open configuration. When the flow of breathable gas is supplied at a pressure above a certain threshold, for example above a therapeutic pressure level such as 6 cmH₂O, the BTA vent may be configured to adopt the closed configuration.

A further explanation of a patient interface system comprising a vent that may be considered to act in the manner of a BTA vent, as described above, is provided in PCT Application No. PCT/US2012/055148, the contents of which are hereby incorporated by reference.

In one application, a BTA vent may be used in a patient interface system in which the BTA vent is configured to adopt the open configuration when the patient first dons the patient interface 3000 and while the patient is detected as being awake by the RPT device 4000. In this configuration, the RPT device may not supply a flow of breathable gas, or may be configured to provide a small flow of breathable gas to help flush out exhaled CO₂ from the plenum chamber 3200. Once the RPT device 4000 detects that the patient has gone to sleep, the flow of breathable gas may be supplied at a therapeutic pressure, which causes the BTA vent to adopt the closed configuration.

### 5.3.5.2.1 Anti-Asphyxia Valve

One form of BTA vent is an anti-asphyxia valve (AAV) which is conventionally used in patient interfaces which cover both the nose and mouth as a measure to mitigate the risk of asphyxiation. The AAV ensures ventilation to the airways of the patient 1000 in case of disruption of the supply of breathable gas to the plenum chamber 3200 and/or the airways of the patient 1000. In certain forms of the present technology, a patient interface 3000 may comprise a conventional design of AAV acting in use as a BTA vent, as described above.

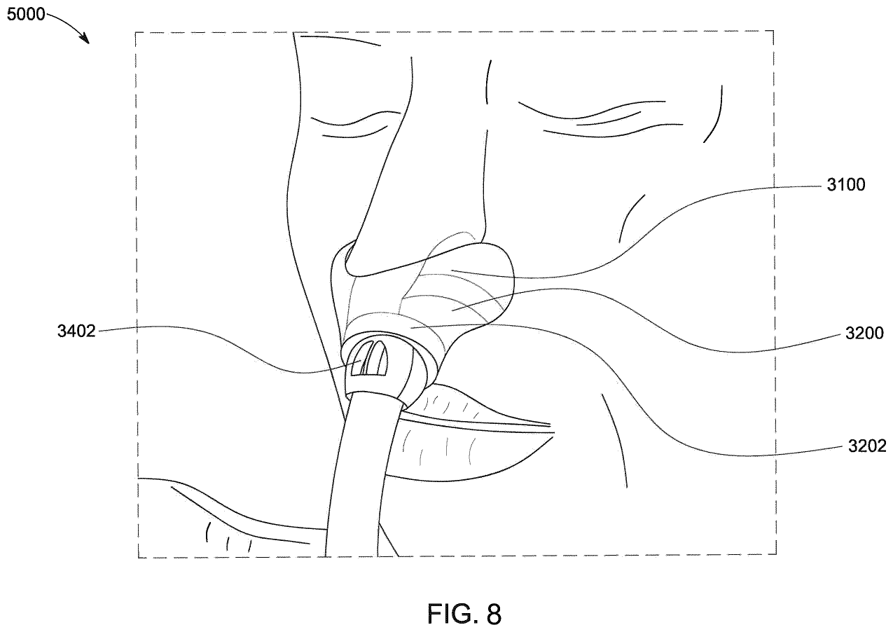

For example, in the form of the technology illustrated in FIG. 8, the patient interface 3000 comprises a vent 3400 that, in use acts as a BTA vent in the manner described above. The BTA vent may be in the form of an anti-asphyxia valve 3402. In the illustrated form of the technology, the anti-asphyxia valve 3402 is located on or proximate an end of the air circuit 4170 that connects to the inlet port 3202 of the plenum chamber 3200. In other forms, the AAV may be located in another location, for example in the plenum chamber 3200, or in a tube connected between the air circuit 4170 and the plenum chamber 3200.

The anti-asphyxia valve 3402 may comprise a flap which, when the valve 3402 is in the closed configuration, is configured to cover an opening in a wall of the air circuit 4170 and to prevent or limit leakage of gas contained in the plenum chamber 3200 in use. When the valve is in the open configuration, for example in case of a disruption or reduction in the flow of the breathable gas to the plenum chamber 3200, or if the pressure of the breathable gas supplied by the RPT device 4000 has not yet ramped up (e.g. if the patient is detected as still being awake), the flap is in a position where the opening is less occluded to allow the patient 1000 to breathe directly to and from the ambient atmosphere.

### 5.3.5.3 Expiratory Resistance Valve

In certain forms of the technology, positive pressure within the plenum chamber 3200 may be created in a manner other than through the supply of air into the plenum chamber 3200 from a RPT device 4000. For example, in one form of respiratory therapy system 2000, positive pressure is created in the plenum chamber 3200 from the flow of gases exhaled by the patient. Such a system may be referred to as an expiratory positive airway pressure (EPAP) system. EPAP systems according to certain forms of the technology may not comprise a RPT device or an air circuit as described herein. Instead, the EPAP system may comprise a vent that is configured to create and maintain the therapeutic pressure in the plenum chamber from the flow of gases exhaled by the patient.

### 5.3.6 Mouth Closure Structure

In certain forms of the technology, the patient interface 3000 comprises a mouth closure structure 3910. The mouth closure structure 3910 is a structure, which may be formed of a component or an assembly of components, that is configured to promote closure of the patient's mouth when the patient interface is worn by the patient.

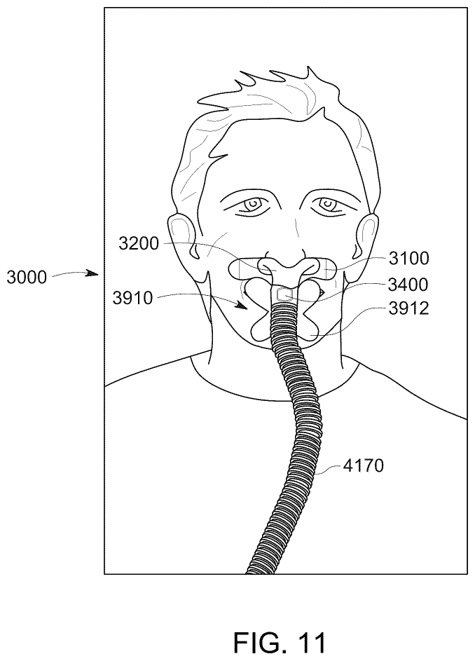

In some forms of the technology, for example those shown in FIGS. 11 to 13, the mouth closure structure 3910 may be comprised as part of a patient interface 3000 that comprises a seal-forming structure 3100 configured to adhere to a patient's face to form a seal. In other forms of the technology, for example those shown in FIGS. 14 to 16, the mouth closure structure 3910 may be comprised as part of a patient interface 3000 that comprises a seal-forming structure 3100 that forms a seal with the patient's face through an alternative mechanism, for example by retention of the seal-forming structure 3100 against the patient's face using headgear.

It should be understood that, even if a form of mouth closure structure 3910 is only described in the context of a patient interface 3000 comprising one type of seal-forming structure 3100, in another form of the technology, the same form of mouth closure structure 3910 may be used in a patient interface 3000 with another type of seal-forming structure 3100, even if that combination is not expressly described.

### 5.3.6.1 Function of Mouth Closure Structure

The mouth closure structure 3910 functions to promote closure of the patient's mouth when the patient interface is worn by the patient and when the patient interface is receiving respiratory therapy.

In certain forms, the mouth closure structure 3910 may be configured to promote closure of the patient's mouth by providing a resistance force to the patient opening their mouth. For example, the structure may be configured such that, when the patient's mouth is closed, little or no force is applied to the patient's mouth but that, if the patient attempts to open their mouth, the resistive force applies and the resistive force tends to prevent or impede opening of the mouth.

In certain forms, the mouth closure structure 3910 may be alternatively or additionally configured to promote closure of the patient's mouth by applying a mouth closure force on a part of the patient's face in order to urge the patient's mouth closed, for example by applying a force on a part of the patient's face near the mouth. In certain examples, the mouth closure structure 3910 may apply a force on the patient's lower jaw in a generally superior direction, urging the lower jaw towards the upper jaw and consequently urging the mouth closed.

In some forms, the mouth closure structure 3910 may be configured to promote closure of the patient's mouth so as to seal the mouth and substantially prevent the flow of gas through the patient's mouth opening. This may occur, for example through the mouth closure structure 3910 acting to urge the patient's lips together sufficiently tightly so as to form a seal. Alternatively (or additionally), this may occur through the mouth closure structure 3910 being positioned across the patient's mouth so as to seal the mouth opening itself.

However, in other forms, the mouth closure structure 3910 does not seal the patient's mouth and it should be understood that reference to the patient's mouth being “closed” does not necessarily require that the patient's mouth is completely closed, i.e. sealed so as to prevent gas passing in and out of the mouth opening. The mouth closure structure 3910 may be configured to prevent the mouth from opening any further than a particular position, which may be near-closed, and/or may be configured to urge the mouth to a more closed, even if not completely closed, state.

In certain forms the mouth closure structure 3910 may be configured to allow the patient to open their mouth, or part thereof, in certain conditions, for example upon exertion of a sufficient degree of force against the mouth closure structure 3910. This may be desirable in case the patient needs to open their mouth to breathe, for example upon a blockage to their nasal air passages, a fault in the RPT device or if the patient panics and has a perceived need to breathe through their mouth for comfort.

### 5.3.6.2 Adhesive Mouth Closure Structure

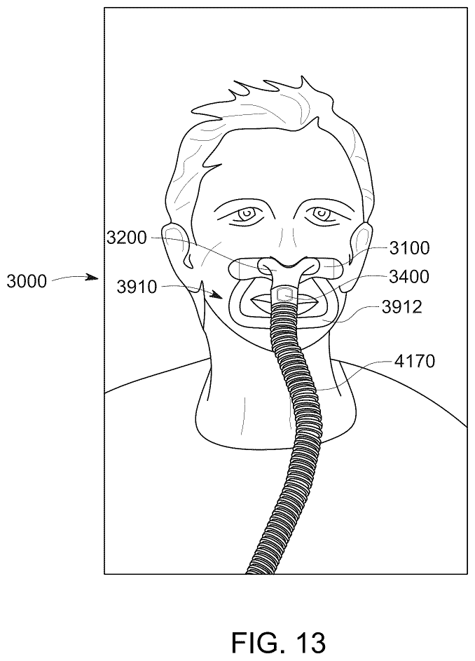

In certain forms of the technology, for example those shown in FIGS. 11 to 16, the mouth closure structure 3910 comprises at least one mouth closure member 3912 comprising an adhesive surface. The adhesive surface is on a side of the mouth closure member 3912 facing towards the patient during use, i.e. a posterior side of the mouth closure member 3912. The adhesive surface of the mouth closure member 3912 is configured in use to adhere to the patient's lips and/or a region of the patient's face proximate the patient's lips. For example, in the form shown in FIGS. 11, 12 and 14 to 16, mouth closure member 3912 adheres to regions of the patient's face proximate their mouth and also to the patient's lips. The mouth closure member 3912 may adhere in use to both inferior and superior lips, or only one of these, depending on the patient's preference and mouth shape. In the exemplary form shown in FIG. 13, the mouth closure member 3912 adheres around the patient's mouth but does not adhere directly to the patient's lips. In each of the forms shown in FIGS. 11 to 16, the mouth closure member 3912 adheres to a region of the patient's face inferior to the mouth and also to a region of the patient's face superior to the mouth. This provides a bracing effect between the upper and lower parts of the mouth, providing resistance against the mouth opening.

In certain forms of the technology, for example those shown in FIGS. 11 to 16, the mouth closure member 3912 is configured so that, when the mouth closure member 3912 is adhered to the patient's face, the mouth closure member 3912 leaves at least a portion of the patient's mouth uncovered. For example, in the case of the patient interface 3000 shown in FIGS. 11, 12 and 13 to 16, when the mouth closure member 3912 is adhered to the patient's face, the side regions of the patient's mouth are left uncovered by the mouth closure member. In the case of the patient interface 3000 shown in FIG. 13, the patient's mouth is left fully uncovered when the mouth closure member 3912 is positioned and adhered to the patient's face. The mouth closure member 3912 may be designed to allow at least a part of the mouth to open during use as a safety measure so that the patient is able to breathe through their mouth if the supply of breathable gas to the nares ceases, or if they feel the urge to do so, without the need to pull against the resistance of the mouth closure member 3912. Leaving at least part of the mouth uncovered may also be a psychological preference for the patient. By leaving the mouth fully uncovered, as in the example of FIG. 13, the patient may be able to talk or consume food or drink while wearing the patient interface 3000.

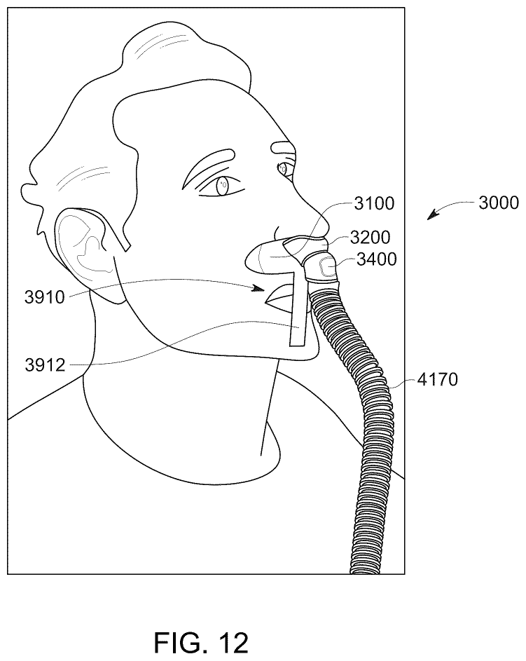

In the forms shown in FIGS. 11, 12 and 13 to 16, the mouth closure members 3912 adhere to the patient's lip superior and lip inferior (see FIGS. 2B to 2D) and span across the patient's mouth. In both forms, the mouth closure members 3912 comprise elongate strips where one end of the strip adheres to the patient's lip superior and the other end of the strip adheres to the lip inferior. In the example of FIG. 11, the mouth closure structure 3910 comprises two mouth closure members 3912 in the form of elongate strips crossing over one another with overlapping central regions (i.e. the mouth closure members are arranged in a cross-shape). In the example of FIG. 12, the mouth closure structure 3910 comprises a single mouth closure member 3912 in the form of an elongate strip with the strip arranged in the superior-inferior direction. In the example of FIGS. 14 to 16, the mouth closure structure 3910 comprises a mouth closure member 3912 in the form of an elongate strip arranged in the superior-inferior direction and with an inferior end of the strip comprising a fork with each arm of the fork extending inferiorly and laterally of the superior part of the strip. In each of these cases, the mouth closure members 3912 cover a central region of the patient's mouth when adhered in position in use.

Forms of the technology in which the mouth closure members 3912 adhere to the patient's mouth and/or regions of the face closely proximate the patient's mouth may be more suitable for use by patients with facial hair than other forms (such as the form shown in FIG. 13) where the mouth closure member 3912 may adhere to regions of the face where, for example, a beard grows. The adhesive may not adhere as effectively to hairy regions of the patient's face, and may be uncomfortable to remove from such regions. In the case of the form shown in FIGS. 14 to 16, the forked shape of the inferior end of the mouth closure structure 3910 may be useful to avoid the inferior tip of the mouth closure member 3912 adhering to a superior, central part of the patient's lip inferior, which may be difficult to adhere to, particularly for patients with facial hair growing in this region. For some patients, they may have less facial hair growing in regions immediately lateral to the superior, central part of the patient's lip inferior.

In the form of the technology shown in FIG. 13, the mouth closure member 3912 is configured to adhere to one or more side-of-mouth regions on one or both lateral sides of the patient's mouth and to a lower lip/chin region between the patient's mouth and chin. In addition, as previously stated, the patient's mouth is left fully uncovered when the mouth closure member 3912 in this form is positioned and adhered to the patient's face. In the example shown, the mouth closure member 3912 may be substantially U-shaped. When the adhesive surface of the mouth closure member 3912 is considered in addition to the adhesive surface 3102 of the seal-forming structure 3100, an adhesive surface entirely surrounds the patient's mouth in this form of the technology.

In certain forms of the technology, the mouth closure structure 3910 may comprise one or more tabs that the patient may use to remove the mouth closure structure 3910. For example, the tabs may be formed as one or more regions of the mouth closure members 3912 where no adhesive is applied on a patient-facing side. Consequently, such region(s) do not adhere to the patient's face and may easily be able to be grasped by the patient to remove the mouth closure member 3912. In one form of the patient interface 3000 shown in FIG. 12, the inferior end of the mouth closure member 3912 may form such a tab, for example. In other forms, the tab may be a region that extends laterally out from a strip-shaped mouth closure member.

The mouth closure member 3912 may be formed from any of the materials and adhesives discussed above in relation to adhesive seal-forming structures 3100.

In certain forms of the technology, the mouth closure structure 3910 may be connected (indirectly or directly) to the seal-forming structure 3100 and/or the plenum chamber 3200. In some forms in which the mouth closure structure 3910 is directly connected to the seal-forming structure 3100 and/or the plenum chamber 3200, the mouth closure structure 3910 may be integrally connected to the seal-forming structure 3100 and/or the plenum chamber 3200. For example, in the exemplary forms shown in FIGS. 11 to 13, the mouth closure members 3912 of the mouth closure structure 3910 may be directly and integrally connected to the seal-forming structure 3100. For example, the seal-forming structure 3100 and the mouth closure members 3912 may be formed from the same sheet of material, for example adhesive tape or any of the materials describe earlier in relation to adhesive seal-forming structures 3100. This may also be the case for a form of the technology closely resembling the form shown in FIG. 14. In other forms, the mouth closure members 3912 may be formed as a separate body to, but be permanently connected to, the seal-forming structure 3100 and/or plenum chamber 3200. Such integral connection may make manufacturing the patient interface 3000 and/or donning the patient interface 3000 easier compared to forms in which the mouth closure structure 3910 is a separate assembly to other components of the patient interface.

In certain forms of the technology, the mouth closure structure 3910 may be adhered to the seal-forming structure 3100 and/or the plenum chamber 3200. For example, a superior region of the mouth closure member 3912 may be adhered to an inferior region of the seal-forming structure 3100 and/or the plenum chamber 3200. In certain forms, a surface of the mouth closure member 3912 facing away from the patient during use may be adhered to a surface of the seal-forming structure 3100 and/or the plenum chamber 3200 facing towards the patient during use. In other forms, a surface of the mouth closure member 3912 facing towards the patient during use may be adhered to a surface of the seal-forming structure 3100 and/or the plenum chamber 3200 facing away from the patient during use.

In some forms of the technology, the mouth closure structure 3910 may be configured to be disconnectable from the seal-forming structure 3100 and/or the plenum chamber 3200. For example, the mouth closure member 3912 may be connected to the seal-forming structure 3100 in a way that the patient is able to readily disconnect the mouth closure member 3912. In one example, perforations may be made at the junction between the mouth closure member 3912 and the seal-forming structure 3100 and the patient may easily tear the mouth closure member 3912 along the line of the perforations. In another example, the mouth closure member 3912 may be joined to the seal-forming structure 3100 by a neck of material that may be easily torn by the patient. The patient interface 3000 may be supplied with the mouth closure structure 3910 connected to the seal-forming structure 3100 and/or the plenum chamber 3200 in such a way so that the patient has the option to easily remove the mouth closure structure 3910 if they prefer not to use it. In some forms, the patient interface 3000 may comprise a plurality of mouth closure members 3912 each configured to be disconnectable from the seal-forming structure 3100 and/or the plenum chamber 3200. In such forms, the patient may select which of the mouth closure members 3912 they leave in place and which (if any) they remove. Consequently, the patient is able to tailor the form of the mouth closure structure 3910 to suit their preferences. For example, the patient interface 3000 may be supplied with any two or more of the forms of mouth closure members 3912 illustrated in FIGS. 11 to 13 being connected to the seal-forming structure 3100 in a way that makes them easy to disconnect.

In the form of the technology illustrated in FIGS. 14 to 16, the mouth closure structure 3910 comprises a mouth closure structure base 3914 and a mouth closure member 3912. The mouth closure member 3912 is connected to the mouth closure structure base 3914, for example the mouth closure member 3912 and the mouth closure structure base 3914 may be integrally formed from the same body of material. The mouth closure structure base 3914 may be configured to connect to the seal-forming structure 3100 and the mouth closure member 3912 may extend inferiorly from the mouth closure structure base 3914 when in use. The mouth closure structure base 3914 may comprise one or more mouth closure structure openings 3916 which may engage with a part of the seal-forming structure 3100 or, in other forms, part of the plenum chamber 3200, to directly connect the mouth closure structure 3910 to the seal-forming structure 3100 and/or plenum chamber 3200. In the form illustrated in FIGS. 14 to 16, the mouth closure structure 3910 may be configured for use with a patient interface 3000 in which the seal-forming structure 3100 comprises nasal prongs. In this form, the mouth closure structure openings 3916 may have a suitable size and shape that the nasal prongs of the seal-forming structure 3100 may protrude through the mouth closure structure openings 3916 in order to retain the mouth closure structure 3910 in position relative to the seal-forming structure 3100. Once assembled in this way, the mouth closure structure base 3914 is positioned around the necks of the nasal prongs in the manner of a collar. In the form illustrated, the mouth closure structure openings 3916 are positioned laterally adjacently and symmetrically in the mouth closure structure base 3914 in order to achieve this. In this form, the mouth closure structure 3910 may be able to be completely removed from the rest of the patient interface 3000 and is able to be replaced, for example after use. In addition, mouth closure structures of different shapes and/or sizes may be provided for use with the same patient interface 3000 so that a patient may select the mouth closure structure 3910 that most suits their needs. While the type of mouth closure structure 3910 shown in FIGS. 14 to 16 is a type in which the mouth closure member 3912 has a forked inferior tip, as described above, in other forms, the mouth closure structure 3910 may have a mouth closure member 3912 with a different shape, for example one of the other types/shapes described herein.

### 5.3.6.3 Other Forms of Mouth Closure Structure

In other forms of the technology, the mouth closure structure 3910 may promote closure of the patient's mouth when the patient interface is worn by the patient in a different way. In some examples, the mouth closure structure 3910 may comprise a chin strap, i.e. a strap that passes under the patient's chin in use. The chin strap may be positioned and configured in order to apply a sufficient force on the chin, for example an inferior surface of the chin, in the superior direction to urge the patient's mouth closed and promote closure of the patient's mouth by making it difficult for the patient to open their mouth. In some forms, the chin strap may be configured so that a force is only exerted on the patient's chin when the patient attempts to open their mouth from a closed position.

In one form, the mouth closure structure 3910 comprises a chin strap having two ends and a central portion located between the two ends. Each end is configured to connect to a respective portion of the patient interface 3000 located laterally, for example portions of the positioning and stabilising structure 3300 that overlays the patient's respective cheek regions. In this way, the mouth closure structure 3910 may be indirectly connected to the seal-forming structure 3100 and to the plenum chamber 3200, i.e. connected via the lateral portions of the patient interface 3000. The central portion may be configured to be positioned under the patient's chin in use. In another form, the two ends of the chin strap may be configured to adhere to the patient's face, for example to opposite cheek regions of the patient's face, in order to anchor them in position during use.

### 5.3.6.4 Benefit of Mouth Closure Structure

It may be advantageous to breathe through the nasal passages as the primary or exclusive channel for breathing when asleep. Various health benefits are considered to result from nasal breathing, and keeping the mouth closed may also prevent snoring.

By keeping the mouth closed when a patient is receiving respiratory treatment, these benefits may be achieved. Also, when the respiratory therapy is delivered to the patient's nasal passages and not their mouth, keeping the patient's mouth closed ensures that the patient is only able to breathe the air delivered during the respiratory therapy, and is not able to breathe ambient air, thereby “short-circuiting” the respiratory therapy.

For those patients who sleep with their mouths open, the options for patient interfaces through which they can receive respiratory therapy may be limited to those which supply breathable gas (e.g. pressurised breathable gas in the case of CPAP therapy) to the mouth so that they are unable to breathe ambient air through their mouths. That is, these patients may be limited to selecting patient interfaces that cover their mouths. Such patient interfaces may be bulkier and more uncomfortable than patient interfaces which only supply air to the nasal passages. In addition, such patient interfaces may make contact with a larger area of the patient's face during use, which may result in a greater amount of skin irritation or facial marking compared to a smaller mask. By including a structure to promote closure the mouth during use of the patient interface, patients in this situation may have a greater choice of patient interfaces that they are able to use effectively. For example, some nasal masks, nasal cradle masks and/or nasal pillow masks may be available for a patient who breathes through their mouth while asleep to use effectively if it incorporates a mouth closure structure as described in this specification.

### 5.4 Air Circuit

In one form, the patient interface 3000 may be comprised as part of a patient interface system 5000 which also includes an air circuit 4170. The air circuit 4170 is configured to convey breathable gas to the patient interface 3000 for delivery to the airways of the patient 1000. For example, the air circuit 4170 of the form of the technology shown in FIG. 1 conveys the breathable gas from the RPT device 4000 to the plenum chamber 3200.

A first end of the air circuit 4170 may be connected to the plenum chamber inlet port 3202. A second end of the air circuit 4170, which may be opposite to the first end, may be connected to an RPT device 4000.

In exemplary forms of the technology, the air circuit 4170 is flexible.

In certain forms, the geometric dimensions of the air circuit 4170 may depend on the flow parameters of the breathable gas supplied to the patient from the RPT device 4000. For instance, the diameter of the air circuit 4170 may be relatively small in the case of an RPT device 4000 configured to provide a supply of breathable gas at relatively low pressures (e.g. 2 to 6 cmH₂O), i.e. low pressure therapy. The diameter of the air circuit 4170 may be relatively larger for use with RPT devices 4000 configured to supply breathable gas at higher pressures (e.g. 6 to 20 cmH₂O).

### 5.4.1 Positioning of Air Circuit

The patient interfaces 3000 described in relation to various forms of the technology herein may be accommodate the positioning of air circuits 4170 in various arrangements with respect to the patient.

In certain forms, the air circuit 4170 may be configured to be routed to the patient interface 3000 from substantially above a transverse plane which is configured to pass through the patient's nasal and/or mouth regions, e.g. the Frankfort horizontal (see FIG. 2E). In such a form, the patient interface 3000 may comprise a positioning structure configured to hold part of the air circuit 4170 in relation to the patient's head in the desired position, for example the part of the air circuit 4170 may be held in a position superior to the patient's otobasion superior. For example, the positioning structure may comprise one or more straps configured to be worn on the patient's head and to engage the air circuit 4170. In one form, the air circuit 4170 may be configured to pass over the top and/or the back of a patient's head. Alternatively, or additionally, the air circuit 4170 may be configured to pass behind and/or close to the back of a patient's neck when the patient interface system 5000 is in use.

In another form, the air circuit 4170 may be configured to reach the patient interface 3000 from substantially below a transverse plane which is configured to pass through the patient's nasal and/or mouth regions, e.g. the Frankfort horizontal. Such an arrangement is shown in FIGS. 3A, 4A, 4B and 10F, for example. In this form, the patient interface 3000 may not need any positioning structure to secure the air circuit 4170 in place.

In one form, the air circuit 4170 may be routed around and/or proximal to one or both ears of the patient 1000. For instance, the air circuit 4170 may branch into two conduits before the end of the air circuit 4170 that connects to the patient interface 3000. Each of the conduits may be configured to be able to be passed behind respective ears of the patient 1000 in use.

### 5.5 Rpt Device

An RPT device 4000 in accordance with one aspect of the present technology comprises mechanical, pneumatic, and/or electrical components and is configured to execute one or more algorithms, such as any of the methods, in whole or in part, described herein. The RPT device 4000 may be configured to generate a flow of air for delivery to a patient's airways, such as to treat one or more of the respiratory conditions described elsewhere in the present document.

In certain forms, the RPT device 4000 may be configured to deliver a flow of air to the patient interface 3000 at a positive pressure with respect to ambient. The RPT device 4000 may be configured to deliver air at a therapeutic pressure, for example at least 6 cmH₂O with respect to ambient. Conventional RPT devices 4000 may be used for this purpose.

In other forms, the RPT device 4000 may be configured to deliver a flow or air to the patient interface 3000 at a lower pressure (but still at a positive pressure relative to ambient), for example 2 to 6 cmH₂O with respect to ambient. Respiratory therapy systems incorporating RPT devices 4000 delivering a flow of air at such pressures may be useful for providing low level therapy. For example, such systems may be useful for treating, or ameliorating snoring, or other mild respiratory conditions. Compared to an RPT device that is able to deliver air at higher pressures, for example RPT devices that may be suitable for treating obstructive sleep apnea, such systems may be cheaper to manufacture, use less power and be more compact in size.

FIG. 1 illustrates a respiratory therapy system 2000 incorporating a RPT device 4000 of the type just described. In the form shown in FIG. 1, breathable gas is conveyed to the patient interface 3000 RPT device 4000 which is compact in size and may therefore be portable, i.e. able to be carried by the patient during use, e.g. mounted on the patient's person or clothing. For example, the RPT device 4000 may be strapped around the patient's neck or arm, or carried in a pocket, in use.

Breathable gas from the RPT device 4000 may be conveyed to the patient interface 3000 through an air circuit 4170. The inlet port 3202 of the plenum chamber 3200 may be connected to an end of the air circuit 4170 with the other end of the air circuit 4170 being connected to the RPT device. Since the flow rate and/or pressure of the supply of air may be lower than with a conventional RPT device 4000 (e.g. a CPAP device), the air circuit 4170 may have a reduced diameter compared to conventional air circuits. For example, in certain forms, the air circuit 4170 may have a diameter in the range 5-15 mm, for example 10 mm. A smaller diameter tube provides more impedance to the flow of air than a larger diameter tube but this is acceptable if the flow rate and/or pressure to be delivered is also low. A smaller diameter tube may be desirable as being less bulky and obtrusive, easier to store or package, and cheaper to manufacture.

### 5.6 Glossary

For the purposes of the present technology disclosure, in certain forms of the present technology, one or more of the following definitions may apply. In other forms of the present technology, alternative definitions may apply.

### 5.6.1 General

Air: In certain forms of the present technology, air may be taken to mean atmospheric air, and in other forms of the present technology air may be taken to mean some other combination of breathable gases, e.g. oxygen enriched air.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB (Sleep Disordered Breathing) events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Oxygen enriched air: Air with a concentration of oxygen greater than that of atmospheric air (21%), for example at least about 50% oxygen, at least about 60% oxygen, at least about 70% oxygen, at least about 80% oxygen, at least about 90% oxygen, at least about 95% oxygen, at least about 98% oxygen, or at least about 99% oxygen. “Oxygen enriched air” is sometimes shortened to “oxygen”.

Medical Oxygen: Medical oxygen is defined as oxygen enriched air with an oxygen concentration of 80% or greater.

Patient: A person, whether or not they are suffering from a respiratory condition.

Pressure: Force per unit area. Pressure may be expressed in a range of units, including cmH₂O, g-f/cm2 and hectopascal. 1 cmH₂O is equal to 1 g-f/cm2 and is approximately 0.98 hectopascal (1 hectopascal=100 Pa=100 N/m2=1 millibar˜0.001 atm). In this specification, unless otherwise stated, pressure is given in units of cmH₂O.

The pressure in the patient interface is given the symbol Pm, while the treatment pressure, which represents a target value to be achieved by the interface pressure Pm at the current instant of time, is given the symbol Pt.

Respiratory Pressure Therapy: The application of a supply of air to an entrance to the airways at a treatment pressure that is typically positive with respect to atmosphere.

Ventilator: A mechanical device that provides pressure support to a patient to perform some or all of the work of breathing.

### 5.6.1.1 Materials

Silicone or Silicone Elastomer: A synthetic rubber. In this specification, a reference to silicone is a reference to liquid silicone rubber (LSR) or a compression molded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45 as measured using ASTM D2240.

Polycarbonate: a thermoplastic polymer of Bisphenol-A Carbonate.

### 5.6.1.2 Mechanical Properties

Resilience: Ability of a material to absorb energy when deformed elastically and to release the energy upon unloading.

Resilient: Will release substantially all of the energy when unloaded. Includes e.g. certain silicones, and thermoplastic elastomers.

Hardness: The ability of a material per se to resist deformation (e.g. described by a Young's Modulus, or an indentation hardness scale measured on a standardised sample size).
‘Soft’ materials may include silicone or thermo-plastic elastomer (TPE), and may, e.g. readily deform under finger pressure.
‘Hard’ materials may include polycarbonate, polypropylene, steel or aluminium, and may not e.g. readily deform under finger pressure.

Stiffness (or rigidity) of a structure or component: The ability of the structure or component to resist deformation in response to an applied load. The load may be a force or a moment, e.g. compression, tension, bending or torsion. The structure or component may offer different resistances in different directions. The inverse of stiffness is flexibility.

Floppy structure or component: A structure or component that will change shape, e.g. bend, when caused to support its own weight, within a relatively short period of time such as 1 second.

Rigid structure or component: A structure or component that will not substantially change shape when subject to the loads typically encountered in use. An example of such a use may be setting up and maintaining a patient interface in sealing relationship with an entrance to a patient's airways, e.g. at a load of approximately 20 to 30 cmH₂O pressure.

As an example, an I-beam may comprise a different bending stiffness (resistance to a bending load) in a first direction in comparison to a second, orthogonal direction. In another example, a structure or component may be floppy in a first direction and rigid in a second direction.

### 5.6.2 Anatomy

### 5.6.2.1 Anatomy of the Face

Ala: the external outer wall or “wing” of each nostril (plural: alar)

Alare: The most lateral point on the nasal ala.

Alar curvature (or alar crest) point: The most posterior point in the curved base line of each ala, found in the crease formed by the union of the ala with the cheek.

Auricle: The whole external visible part of the ear.

(nose) Bony framework: The bony framework of the nose comprises the nasal bones, the frontal process of the maxillae and the nasal part of the frontal bone.

(nose) Cartilaginous framework: The cartilaginous framework of the nose comprises the septal, lateral, major and minor cartilages.

Columella: the strip of skin that separates the nares and which runs from the pronasale to the upper lip.

Columella angle: The angle between the line drawn through the midpoint of the nostril aperture and a line drawn perpendicular to the Frankfort horizontal while intersecting subnasale.

Frankfort horizontal plane: A line extending from the most inferior point of the orbital margin to the left tragion. The tragion is the deepest point in the notch superior to the tragus of the auricle.

Glabella: Located on the soft tissue, the most prominent point in the midsagittal plane of the forehead.

Lateral nasal cartilage: A generally triangular plate of cartilage. Its superior margin is attached to the nasal bone and frontal process of the maxilla, and its inferior margin is connected to the greater alar cartilage.

Lip, lower (labrale inferius):

Lip, upper (labrale superius):

Greater alar cartilage: A plate of cartilage lying below the lateral nasal cartilage. It is curved around the anterior part of the naris. Its posterior end is connected to the frontal process of the maxilla by a tough fibrous membrane containing three or four minor cartilages of the ala.

Nares (Nostrils): Approximately ellipsoidal apertures forming the entrance to the nasal cavity. The singular form of nares is naris (nostril). The nares are separated by the nasal septum.

Naso-labial sulcus or Naso-labial fold: The skin fold or groove that runs from each side of the nose to the corners of the mouth, separating the cheeks from the upper lip.

Naso-labial angle: The angle between the columella and the upper lip, while intersecting subnasale.

Otobasion inferior: The lowest point of attachment of the auricle to the skin of the face.

Otobasion superior: The highest point of attachment of the auricle to the skin of the face.

Pronasale: the most protruded point or tip of the nose, which can be identified in lateral view of the rest of the portion of the head.

Philtrum: the midline groove that runs from lower border of the nasal septum to the top of the lip in the upper lip region.

Pogonion: Located on the soft tissue, the most anterior midpoint of the chin.

Ridge (nasal): The nasal ridge is the midline prominence of the nose, extending from the Sellion to the Pronasale.

Sagittal plane: A vertical plane that passes from anterior (front) to posterior (rear). The midsagittal plane is a sagittal plane that divides the body into right and left halves.

Sellion: Located on the soft tissue, the most concave point overlying the area of the frontonasal suture.

Septal cartilage (nasal): The nasal septal cartilage forms part of the septum and divides the front part of the nasal cavity.

Subalare: The point at the lower margin of the alar base, where the alar base joins with the skin of the superior (upper) lip.

Subnasal point: Located on the soft tissue, the point at which the columella merges with the upper lip in the midsagittal plane.

Supramenton: The point of greatest concavity in the midline of the lower lip between labrale inferius and soft tissue pogonion

### Anatomy of the Skull

Frontal bone: The frontal bone includes a large vertical portion, the squama frontalis, corresponding to the region known as the forehead.

Mandible: The mandible forms the lower jaw. The mental protuberance is the bony protuberance of the jaw that forms the chin.

Maxilla: The maxilla forms the upper jaw and is located above the mandible and below the orbits. The frontal process of the maxilla projects upwards by the side of the nose, and forms part of its lateral boundary.

Nasal bones: The nasal bones are two small oblong bones, varying in size and form in different individuals; they are placed side by side at the middle and upper part of the face, and form, by their junction, the “bridge” of the nose.

Nasion: The intersection of the frontal bone and the two nasal bones, a depressed area directly between the eyes and superior to the bridge of the nose.

Occipital bone: The occipital bone is situated at the back and lower part of the cranium. It includes an oval aperture, the foramen magnum, through which the cranial cavity communicates with the vertebral canal. The curved plate behind the foramen magnum is the squama occipitalis.

Orbit: The bony cavity in the skull to contain the eyeball.

Parietal bones: The parietal bones are the bones that, when joined together, form the roof and sides of the cranium.

Temporal bones: The temporal bones are situated on the bases and sides of the skull, and support that part of the face known as the temple.

Zygomatic bones: The face includes two zygomatic bones, located in the upper and lateral parts of the face and forming the prominence of the cheek.

### 5.6.3 Patient Interface

Anti-asphyxia valve (AAV): The component or sub-assembly of a mask system that, by opening to atmosphere in a failsafe manner, reduces the risk of excessive CO₂ rebreathing by a patient.

Elbow: An elbow is an example of a structure that directs an axis of flow of air travelling therethrough to change direction through an angle. In one form, the angle may be approximately 90 degrees. In another form, the angle may be more, or less than 90 degrees. The elbow may have an approximately circular cross-section. In another form the elbow may have an oval or a rectangular cross-section. In certain forms an elbow may be rotatable with respect to a mating component, e.g. about 360 degrees. In certain forms an elbow may be removable from a mating component, e.g. via a snap connection. In certain forms, an elbow may be assembled to a mating component via a one-time snap during manufacture, but not removable by a patient.

Frame: Frame will be taken to mean a mask structure that bears the load of tension between two or more points of connection with a headgear. A mask frame may be a non-airtight load bearing structure in the mask. However, some forms of mask frame may also be air-tight.

Membrane: Membrane will be taken to mean a typically thin element that has, preferably, substantially no resistance to bending, but has resistance to being stretched.

Plenum chamber: a mask plenum chamber will be taken to mean a portion of a patient interface having walls at least partially enclosing a volume of space, the volume having air therein pressurised above atmospheric pressure in use. A shell may form part of the walls of a mask plenum chamber.

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Shell: A shell will be taken to mean a curved, relatively thin structure having bending, tensile and compressive stiffness. For example, a curved structural wall of a mask may be a shell. In some forms, a shell may be faceted. In some forms a shell may be airtight. In some forms a shell may not be airtight.

Stiffener: A stiffener will be taken to mean a structural component designed to increase the bending resistance of another component in at least one direction.

Strut: A strut will be taken to be a structural component designed to increase the compression resistance of another component in at least one direction.

Swivel (noun): A subassembly of components configured to rotate about a common axis, preferably independently, preferably under low torque. In one form, the swivel may be constructed to rotate through an angle of at least 360 degrees. In another form, the swivel may be constructed to rotate through an angle less than 360 degrees. When used in the context of an air delivery conduit, the sub-assembly of components preferably comprises a matched pair of cylindrical conduits. There may be little or no leak flow of air from the swivel in use.

Tie (noun): A structure designed to resist tension.

Vent: (noun): A structure that allows a flow of air from an interior of the mask, or conduit, to ambient air for clinically effective washout of exhaled gases. For example, a clinically effective washout may involve a flow rate of about 10 litres per minute to about 100 litres per minute, depending on the mask design and treatment pressure.

### 5.7 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

### 5.8 Reference Signs List

1000 Patient
3132 Septum Region
3141 Alar Rim Region
3142 Superior-most Region of Lip Superior
3143 Anterior Region of the Nose Inferior to the Pronasale
3144 Region Inferior to the Alar Crest Point
2000 Respiratory Therapy System
3000 Patient Interface
3100 Seal-Forming Structure
3102 Adhesive Surface
3132 Septum Region
3150 Cushion Module
3200 Plenum Chamber
3202 Plenum Chamber Inlet Port
3250 Nasal Prongs
3300 Positioning and Stabilising Structure
3310 Strap
3320 Tab
3350 Tube
3362 Extendable Concertina Structure
3363 Non-Extendable Tube Sections
3400 Vent
3402 Anti-Asphyxia Valve
3600 Connection Port
3610 Elbow
3700 Forehead Support
3910 Mouth Closure Structure
3912 Mouth Closure Member
3914 Mouth Closure Structure Base
3916 Mouth Closure Structure Opening
4000 RPT device
4170 Air Circuit
5000 Patient Interface System

1. A patient interface for use in delivering breathable gas to a patient, the patient interface comprising:
a plenum chamber pressurisable to a therapeutic pressure of at least 6 cmH₂O above ambient air pressure, said plenum chamber including a plenum chamber inlet port configured to receive a flow of breathable gas at the therapeutic pressure for breathing by the patient;
a seal-forming structure provided to the plenum chamber, wherein the seal-forming structure is configured to form a seal with a region of the patient's face surrounding an entrance to the patient's nares but not around the patient's mouth, said seal-forming structure having an opening therein such that the flow of breathable gas is delivered to the entrance to the patient's nares, the seal-forming structure being configured to maintain said therapeutic pressure in the plenum chamber throughout the patient's respiratory cycle in use, wherein the seal-forming structure comprises at least one adhesive surface configured in use to adhere to a region of the patient's face to form the seal;
a vent structure to allow a continuous flow of gases exhaled by the patient from an interior of the plenum chamber to ambient, said vent structure being configured to maintain the therapeutic pressure in the plenum chamber in use; and
a mouth closure structure configured to promote closure of the patient's mouth, wherein the mouth closure structure is connected to the seal-forming structure and/or the plenum chamber, and wherein the mouth closure structure comprises a strip or strap configured to engage the patient's mouth and/or chin regions.

2. A patient interface as claimed in claim 1, wherein the mouth closure structure comprises a mouth closure member comprising an adhesive surface configured in use to adhere to the patient's lips and/or a region of the patient's face proximate the patient's lips.

3. A patient interface as claimed in claim 2, wherein the mouth closure member is configured to adhere to a region of the patient's face inferior to the mouth and to a region of the patient's face superior to the mouth.

4. A patient interface as claimed in claim 2, wherein, when the mouth closure member is adhered to the patient's face, the mouth closure member leaves at least a portion of the patient's mouth uncovered.

5. A patient interface as claimed in claim 4, wherein, when the mouth closure member is adhered to the patient's face, the mouth closure member leaves the patient's mouth fully uncovered.

6. A patient interface as claimed in claim 2, wherein the mouth closure member is configured to adhere to one or more side-of-mouth regions on one or both lateral sides of the patient's mouth and to a lower lip/chin region between the patient's mouth and chin.

7. A patient interface as claimed in claim 2, wherein the mouth closure member is configured to span across the patient's mouth.

8. A patient interface as claimed in claim 1, wherein the mouth closure structure is integrally connected to the seal-forming structure and/or the plenum chamber.

9. A patient interface as claimed in claim 1, wherein the mouth closure structure is configured to be disconnectable from the seal-forming structure and/or the plenum chamber.

10. A patient interface for use in delivering breathable gas to a patient, the patient interface comprising:
a plenum chamber pressurisable to a therapeutic pressure of at least 6 cmH₂O above ambient air pressure, said plenum chamber including a plenum chamber inlet port configured to receive a flow of breathable gas at the therapeutic pressure for breathing by the patient;
a seal-forming structure provided to the plenum chamber, wherein the seal-forming structure is configured to form a seal with a region of the patient's face surrounding an entrance to the patient's nares but not around the patient's mouth, said seal-forming structure having an opening therein such that the flow of breathable gas is delivered to the entrance to the patient's nares, the seal-forming structure being configured to maintain said therapeutic pressure in the plenum chamber throughout the patient's respiratory cycle in use;
a vent structure to allow a continuous flow of gases exhaled by the patient from an interior of the plenum chamber to ambient, said vent structure being configured to maintain the therapeutic pressure in the plenum chamber in use; and
a mouth closure structure configured to promote closure of the patient's mouth, wherein the mouth closure structure is connected to the seal-forming structure and/or the plenum chamber, and wherein the mouth closure structure comprises a strip or strap configured to engage the patient's mouth and/or chin regions.

11. A patient interface as claimed in claim 10, wherein the seal-forming structure comprises at least one adhesive surface configured in use to adhere to a region of the patient's face to form the seal.

12. A patient interface as claimed in claim 10, wherein the patient interface further comprises headgear configured to hold the seal-forming structure in sealing position on the patient's face in use.

13. A patient interface as claimed in claim 12, wherein the headgear comprises one or more straps configured to hold the seal-forming structure in sealing position on the patient's face in use.

14. A patient interface as claimed in claim 10, wherein the mouth closure structure comprises a mouth closure member comprising an adhesive surface configured in use to adhere to the patient's lips and/or a region of the patient's face proximate the patient's lips.

15. A patient interface as claimed in claim 14, wherein the mouth closure member is configured to adhere to a region of the patient's face inferior to the mouth and to a region of the patient's face superior to the mouth.

16. A patient interface as claimed in claim 14, wherein, when the mouth closure member is adhered to the patient's face, the mouth closure member leaves at least a portion of the patient's mouth uncovered.

17. A patient interface as claimed in claim 16, wherein, when the mouth closure member is adhered to the patient's face, the mouth closure member leaves the patient's mouth fully uncovered.

18. A patient interface as claimed in claim 15, wherein the mouth closure member is configured to adhere to one or more side-of-mouth regions on one or both lateral sides of the patient's mouth and to a lower lip/chin region between the patient's mouth and chin.

19. A patient interface as claimed in claim 15, wherein the mouth closure member is configured to span across the patient's mouth.

20. A patient interface as claimed in claim 10, wherein the mouth closure structure is configured to be disconnectable from the seal-forming structure and/or the plenum chamber.
